(19) United States
(12) Patent Application Publication
(10) Pub. No.: US 2008/0044428 A1
(43) Pub. Date: Feb. 21, 2008

(54) Glycosylphosphatidylinositol Glycan Signalling Via Integrins Functioning as Glycan Specific Receptors

(75) Inventor: Louis Schofield, Gisborne (AU)

Correspondence Address: SCULLY SCOTT MURPHY & PRESSER, PC, 400 GARDEN CITY PLAZA, SUITE 300, GARDEN CITY, NY, 11530, US

(73) Assignee: THE WALTER AND ELIZA HALL INSTITUTE OF MEDICAL RES, Parkville (AU)

(21) Appl. No.: 11/629,256

(22) PCT Filed: Jun. 10, 2005
(86) PCT No.: PCT/AU05/00842

§ 371 Date: Feb. 16, 2007

(30) Foreign Application Priority Data
Jun. 10, 2004 (AU) 2004903183

(51) Int. Cl. A61K 39/395; A61K 38/16; A61P 43/00; C12N 5/02; G01N 33/92

(52) U.S. Cl. 424/172.1; 435/375; 436/71; 514/12; 514/8

(57) ABSTRACT

The present invention relates generally to a method of modulating integrin-mediated cellular activity and to agents useful for same. More particularly, the present invention contemplates a method of modulating ab integrin-mediated cellular activity by modulating GPI-related signalling. The method of the present invention is useful, inter alia, in the treatment and/or prophylaxis of conditions characterised by aberrant, unwanted or otherwise inappropriate integrin-mediated cellular activity. The present invention is further directed to methods for identifying and/or designing agents capable of modulating the subject integrin dependent signalling mechanism.

### FIELD OF THE INVENTION

The present invention relates generally to a method of modulating integrin-mediated cellular activity and to agents useful for same. More particularly, the present invention contemplates a method of modulating αβ integrin-mediated cellular activity by modulating GPI-related signalling. The method of the present invention is useful, inter alia, in the treatment and/or prophylaxis of conditions characterised by aberrant, unwanted or otherwise inappropriate integrin-mediated cellular activity. The present invention is further directed to methods for identifying and/or designing agents capable of modulating the subject integrin dependent signalling mechanism.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia.

Glycosylphosphatidylinositols (GPIs) are a class of glycolipid common to all eukaryotes (McConville M J, et at., (1993) Biochem. J., 294, 305). They are structurally related to phosphatidylinositol (PI) being composed of PI linked glycosidically to the evolutionarily conserved core glycan sequence Manα1-2Manα1-6Manα1-4GlcN. This tetrasaccharide core glycan may be further substituted with sugars, phosphates and ethanolamine groups in a species and tissue-specific manner. GPI fatty acid moieties can be either diacylglycerols, alkylacylglycerols, monoalkylglycerols or ceramides, with additional lipid modifications to the inositol ring. The overall picture is of a closely related family of glycolipids sharing certain core features but with a high level of variation in fatty acid composition and side-chain modifications to the conserved core glycan.

GPIs can occur either linked to the C-terminus of many different protein species, or “free” in the outer leaflet of the cell membrane. The function of free GPIs remains unclear. The predominant view of GPI function is that this class of molecule serves as a novel form of membrane anchor for proteins (Ferguson M A J (1992) Biochem Soc Trans. 20, 243). However, some studies in the late 1980s and early mid 1990s appeared to provide indirect to circumstantial evidence implicating GPI-derived inositolphosphoglycans (IPGs) as post-receptor mediators of hormone action (Saltiel A R, et al., (1983) Science 233, 967, Saltiel A R, et al., (1987) Biochem. Biophys. Res. Commun., 149, 1084, Saltiel A R, (1991) J. Bioenerg. Biomemb., 23, 29, Chan B L, et al., (1989) Proc. Natl. Acad. Sci. USA, 86, 1756, Repressa J, et al., (1991) Proc. Natl. Acad. Sci. USA, 88, 8016, Vivien D, et al., (1993) J. Cell. Physiol., 155, 437, Eardley D D, et al., (1991) Science, 251, 78, Merida I, et al, (1990) Proc. Natl. Acad. Sci. USA, 87, 9421, Martiny L, et al., (1990) Cell. Signal, 2, 21, Fanjul L F, et al., (1993) J. Cell. Physiol., 155,273, Devemy E, et al., (1994) Cell. Signal., 6, 523). According to this view, binding of hormones to their cognate receptors might result in activation of one or more undefined phospholipase(s) which hydrolyse cell-surface GPIs to release IPG. The IPG is proposed to act as an extracellular “second messenger” mediating several aspects of post-receptor insulin signalling. This view was and is still considered heterodox by many authorities and is not accepted by the majority of researchers. No structure of any hormone/cytokine-sensitive mammalian GPIs has yet to be published; indeed, the proposition that these putative second messengers are GPI-derived IPGs itself remains speculative. The studies largely date from a decade ago and current research does not support this view. Accordingly, there remains considerable uncertainty and indeed scepticism in the scientific community concerning both the role of GPIs as signalling agents and their possible mechanism of action, and indeed the overall significance of this class of molecule.

The predominant view of GPI anchor function is that they serve as a novel form of membrane anchor for proteins (Ferguson M A J (1992) Biochem Soc Trans. 20, 243) and function as a sorting signal for raft association. GPI-anchored proteins are localized within highly specialised microdomains at the cell surface (known as “rafts”, “caveolar complexes”, “detergent-resistant membranes (DRMs)” “glycolipid-enriched membranes (GEMs)” “detergent-insoluble glycolipid-enriched domains (DIGs)” etc). These structures have unusual lipid compositions, enriched in sphingolipids and proteins such as caveolin and β-integrins (Mayor S, et al., (1994) Science, 264, 1948, Lisanti M P, et al. (1995) Mol. Mem. Biol., 12, 121, Bohulslav J, et al., (1995) J. Exp. Med., 181, 1381) and a host of signalling molecules, which are thought to represent dedicated signal transducing complexes. Nonetheless, a number of GPI-anchored proteins do have significant signal transduction capacity within cells i.e. when perturbed they lead to definite biological responses. The consensus is that this occurs through one or both of two possible mechanisms (not mutually exclusive):
(i) the GPI-anchored protein may through the protein domain become associated with one or more authentic “signalling partner” within rafts i.e. physiologically appropriate perturbation of the GPI-anchored protein causes an association with another protein molecule(s) (having specificity for the protein component of the GPI-anchored “initiator”), but which itself has a transmembrane domain able to initiate signal transduction. This is shown schematically in FIG. 1; or
(ii) that GPI-anchored proteins are in extremely small raft structures comprising only a few molecules, but when cross-linked or physiologically perturbed these mini-rafts coalesce to form much larger rafts which then allows the conjunction of signalling molecules in the intracellular region.

In both these models, the role of the GPI has nothing to do with direct signal transduction or interaction with other signalling partners by the glycolipid itself: the GPI simply serves to locate appropriate proteins to rafts and signalling is effected by other processes.

Nevertheless, over the years it has been shown that GPIs of protozoal origin are able to initiate signalling processes when added directly to mammalian cells as exogenous agonists. Example include the regulation of gene expression of many pro-inflammatory loci in macrophages and vascular endothelial cells (Almeida, I. C. and Gazzinelli, R. T., (2001) J. Leukocyle Biol. 70. 467).

There are two competing views on how GPIs of protozoal origin may interact with receptors to transduce signals. The currently unchallenged and widely held view is that the bioactivity of protozoal GPIs in target mammalian cells results simply from their detection by members of the Toll-Like Receptor (TLR) family. The innate immune system can sense the presence of invading pathogens by the use of specific receptors that recognize microbial “pathogen associated molecular patterns” (Schofield, supra), and activation of these receptors initiates host responses. These responses have been shown to be mediated by an ancient family of host membrane proteins known as the Toll-like receptors (TLRs), which recruit and activate signalling molecules involved in innate immunity (Schofield, supra). The human genome encodes at least ten TLR family members (Schofield, supra) which recognise, lipid, protein and nucleic acid products from a variety of pathogens (Tachado supra, Tachado, supra). To date, the only reported explanation for signalling mediated by exogenous protozoal GPIs added to target host cells comes from a study demonstrating GPI recognition by TLR-2 (Campos, M. A. et al., (2002) J. Immunol 167: 416), the Toll-Like Receptor most clearly dedicated to recognition of microbial glycolipids (along with similar activity by TLR-4). Accordingly, TLRs are currently widely believed to be responsible for mediating the bioactivity of GPIs.

In light of the fact that modulating cellular functional activity in a directed manner remains an extremely sought after means of prophylactically and/or therapeutically treating many disease conditions, there is a significant need to fully elucidate both the mechanisms by which cellular functional activity is regulated and means by which these mechanisms can be modulated.

In work leading up to the present invention, it has been surprisingly determined that contrary to current dogma, GPIs do in fact play a very significant role as a signalling agent in the context of integrin-mediated cellular activity. Further, the mechanisms by which GPIs achieve this outcome does not correlate with the signalling mechanism models proposed by the few groups which have postulated that such a mechanism might exist. Specifically, it has been determined that the signalling mechanism of intact GPIs (i.e. those comprising a glycan component and a fatty amino component) is a two-signal mechanism. In this “two-signal” model (which does not preclude additional signals) the GPI glycan binds to integrins which function as glycan-specific receptors (FIG. 13). These may either be originally located within “rafts” or may translocate to these structures after binding to GPI glycans. Binding of the glycan initiates a signalling process involving src-kinases and members of the MAP kinase cascade. Following binding, a lipidated GPI may also be hydrolysed by phospholipases to generate lipidic second messengers which act both independently and in synergy with integrin-mediated signals to promote downstream metabolic and gene expression endpoints (FIG. 13).

The elucidation of this cellular signalling mechanism has now facilitated the development of methodology directed to modulating integrin-mediated cellular activity by regulating either the interaction of GPI with integrin or the signalling-related events which flow therefrom.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

One aspect of the present invention is directed to a method for regulating integrin-mediated cellular activity, said method comprising modulating the functional interaction of a GPI with an integrin wherein inducing or otherwise agonising said interaction upregulates said cellular activity and inhibiting or otherwise antagonising said interaction downregulates said cellular activity.

There is also provided a method for regulating αβ-integrin-mediated cellular activity, said method comprising modulating the functional interaction of a GPI with said αβ-integrin wherein inducing or otherwise agonising said interaction upregulates said cellular activity and inhibiting or otherwise antagonising said interaction downregulates said cellular activity.

There is provided a method for potentiating cytokine signal transduction, said method comprising modulating the functional interaction of a GPI with an integrin, which integrin is expressed on the same cell as the receptor for said cytokine, wherein inducing or otherwise agonising said interaction potentiates said cytokine signal transduction.

There is provided a method for potentiating insulin signal transduction, said method comprising modulating the functional interaction of a GPI with an integrin, which integrin is expressed on the same cell as the receptor for said insulin, wherein inducing or otherwise agonising said interaction potentiates said insulin signal transduction.

The present invention more preferably provides a method for regulating α⊕-integrin-mediated cellular activity, said method comprising modulating the functional interaction of an intact GPI with an integrin wherein inducing or otherwise agonising said interaction upregulates said cellular activity and inhibiting or otherwise antagonising said interaction downregulates said cellular activity.

In another preferred embodiment there is provided a method for regulating αβ-integrin-mediated cellular activity, said method comprising modulating the functional interaction of a GPI inositolglycan domain with an integrin wherein inducing or otherwise agonising said interaction upregulates said cellular activity and inhibiting or otherwise antagonising said interaction downregulates said cellular activity.

Another aspect of the present invention is directed to a method for the treatment and/or prophylaxis of a condition characterised by aberrant integrin-mediated cellular activity, said method comprising modulating the functional interaction of a GPI with an integrin wherein inducing or otherwise agonising said interaction upregulates said cellular activity and inhibiting or otherwise antagonising said interaction downregulates said cellular activity.

More particularly the present invention is directed to a method for the treatment and/or prophylaxis of a condition characterised by aberrant αβ-integrin mediated cellular activity, said method comprising modulating the functional interaction of a GPI with an integrin wherein inducing or otherwise agonising said interaction upregulates said cellular activity and inhibiting or otherwise antagonising said interaction downregulates said cellular activity.

In one preferred embodiment the present invention is directed to a method for the treatment and/or prophylaxis of type II diabetes, said method comprising modulating the functional interaction of a GPI with an αβ-integrin, which integrin is expressed on the same cells as the insulin receptor, wherein inducing or otherwise agonising said interaction potentiates insulin signal transduction.

In another preferred embodiment, the present invention is directed to a method for therapeutically and/or prophylactically treating a prion-related neurodegenerative condition, said method comprising modulating the functional interaction of said prion GPI with an αβ-integrin wherein antagonising said interaction downregulates prion related catalysis of the conversion of native proteins to an aberrant form.

In yet another further aspect, the present invention contemplates a pharmaceutical composition comprising the modulatory agent as hereinbefore defined together with one or more pharmaceutically acceptable carriers and/or diluents. Yet another aspect of the present invention relates to the agent as hereinbefore defined, when used in the method of the present invention.

Still another aspect of the present invention provides a method for detecting an agent capable of modulating the interaction of a GPI with an integrin or its functional equivalent or derivative thereof said method comprising contacting a test system containing said GPI and/or integrin or its functional equivalent or derivative with a putative agent and screening for modulated functional interaction.

### BRIEF DESCRIPTION OF THE DRAWINGS

FIG. 1 shows a schematic representation of two competing views on mechanisms by which GPI-anchored proteins may transduce signals into cells The conventional interpretation holds that the protein component of a GPI-anchored protein has binding specificity for a signalling partner with a transmembrane domain and cytoplasmic domain capable of signal transduction (shown on the right). We additionally propose that the GPI anchors themselves bind to integrins which transduce signals into cells (shown on left). In addition, similar signalling may occur through binding to integrins of “free” cell surface GPIs and GPIs of exogenous origin.

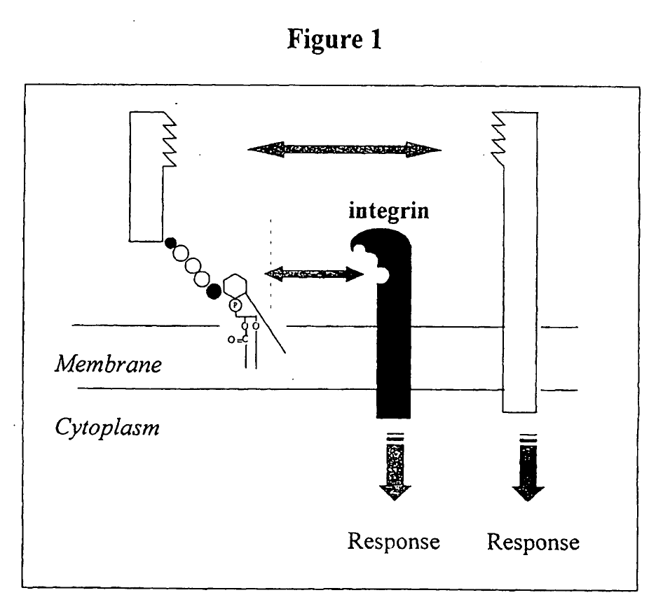

FIG. 2 shows a schematic of the synthetic glycan specified.

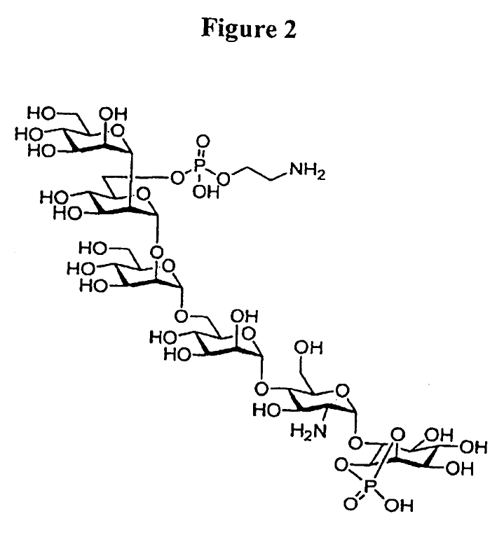

FIG. 3 shows the method used to conjugate the glycan to the BSA carrier protein. Sham conjugation procedures were also followed substituting cysteine for glycan.

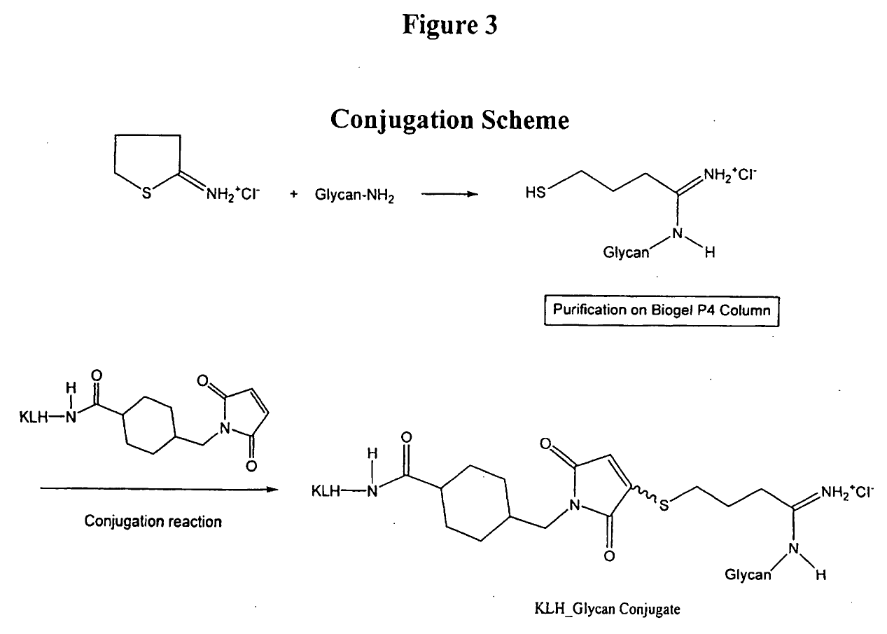

FIG. 4 displays histograms of showing the binding of fluoresceinated BSA-GPI-glycan to CHO cells transfected with αMβ2-integrin, also known as CR3 or Mac-1 (11.5%), and low levels of binding of fluoresceinated BSA-cysteine (0.69%), as well as low levels of binding of both constructs to sham-transfected CHO cells (CHO-Neo), as detected by FACS analysis. The slight increase of binding of fluoresceinated BSA-GPI-glycan to CHO-Neo cells as shown in the second panel (0.4% compared to 5.08%) may reflect binding to constitutively expressed non Mac-1 integrins in CHO-Neo cells.

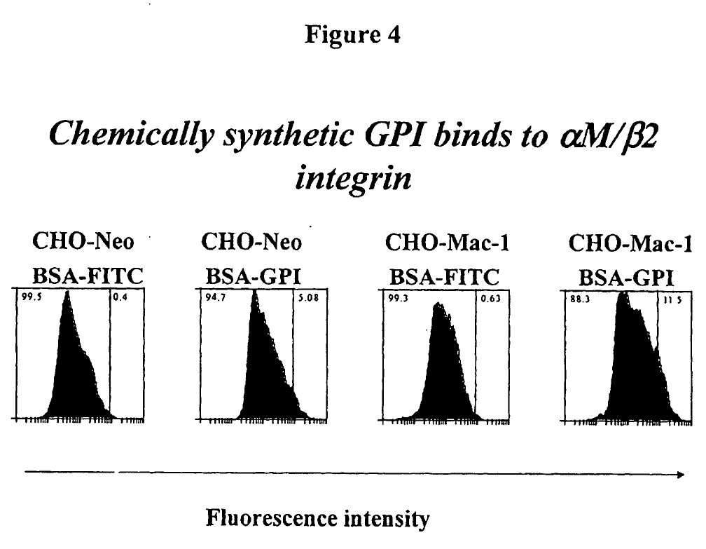

FIG. 5 shows the ability of GPI at different stages in purification to activate ERK in CHO-Mac1 cells compared with CHO-Neo controls as determined by Western blot with phospho-ERK-specific antibodies. Anisomycin is used as an ERK activation control.

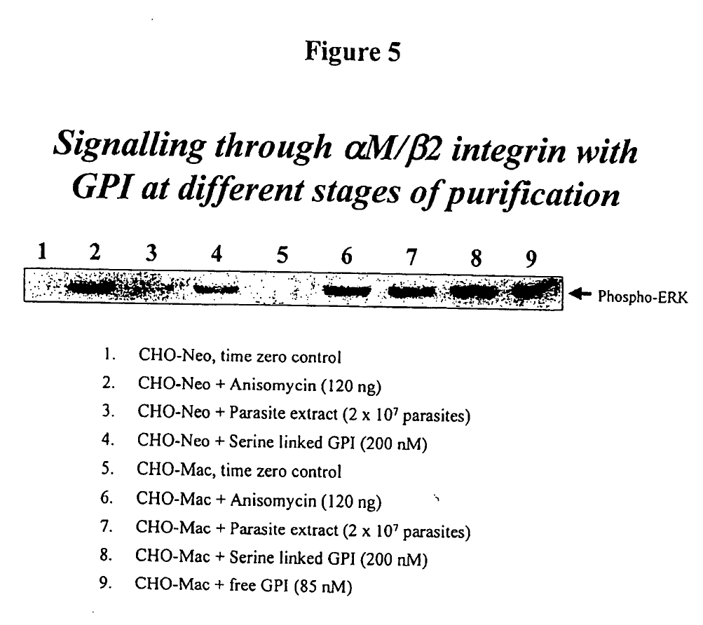

FIG. 6 shows a time-course of activation of MEK and PKC by purified native GPI in CHO-Mac1 cells compared with CHO-Neo controls as determined by Western blot with phospho-MEK- and phospho-PKC-specific antibodies.

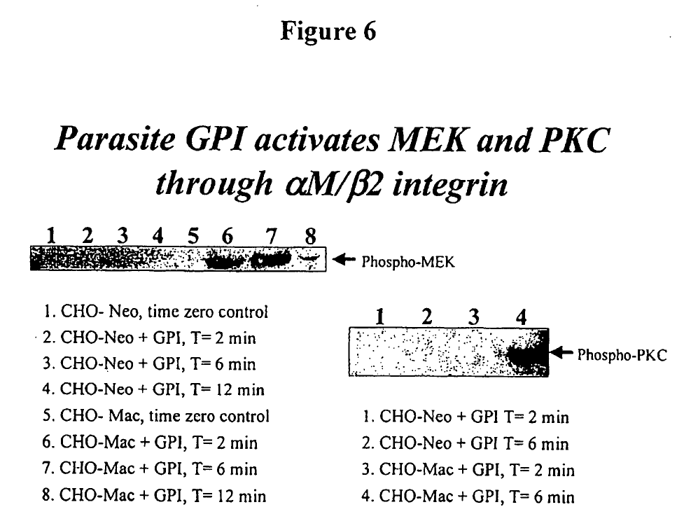

FIG. 7 shows a dose response of free GPI and GPI derived from GPI-anchored protein by exhaustive pronase digestion (scrine-linked) GPI) in activation of ERK in CHO Mac1 cells compared with CHO-Neo controls as determined by Western blot with phospho-ERK-specific antibodies.

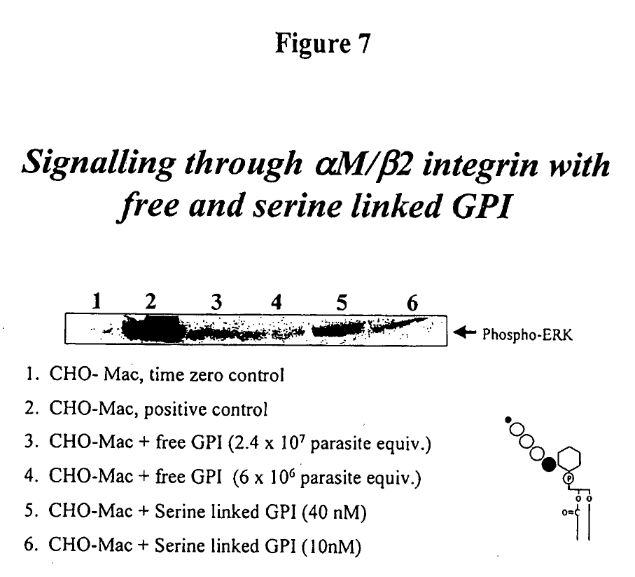

FIG. 8 shows that CHO-Mac1 cells undergo rapid cytoskeletal rearrangements with formation or microfilaments and pseudopodia within 10 minutes of exposure to 100 nM GPI.

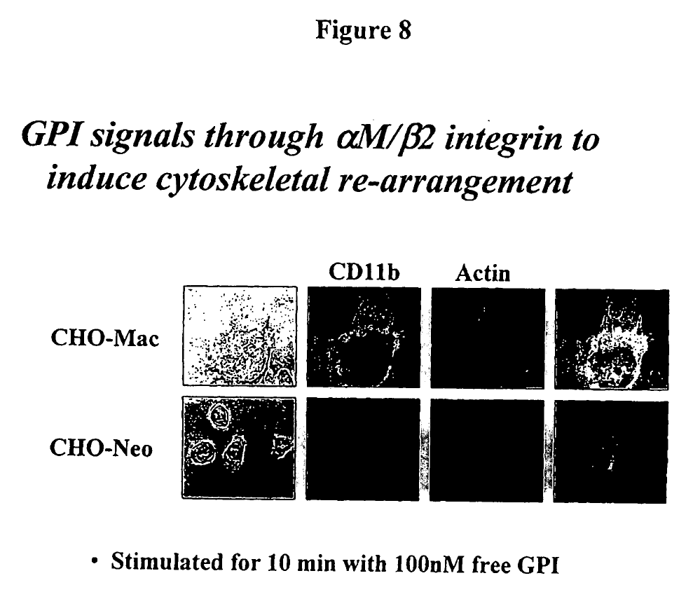

FIG. 9 shows CHO-Mac1 cells exposed to GPI (bottom two panels), or medium (top panel), for 10 minutes and then fixed and stained with FITC-anti-integrin antibodies (CD18) or phalloidin (red). Clearly, as compared to cells exposed to medium, cells exposed to GPI show a much more pronounced punctuate pattern of Mac1 distribution, consistent with re-localization of the integrin after ligand binding.

FIG. 10 lists currently knows integrin chains.

FIG. 11 shows currently understood association of α and β integrin chains.

FIG. 12 is a schematic showing some of the downstream signalling processes activated upon integrin-ligand binding.

FIG. 13 is a schematic outlining our two-signal model of GPI activity following binding integrins.

FIG. 14 is a schematic representation of the synthesis of glycan. 1. Reagents: a. 4, AgOTf, NIS, CH₂Cl₂/Et₂O (38% a); b. NaOMe, CH₂Cl₂/MeOH (83%); c. 6, TMSOTf, CH₂Cl₂ (75%); d. NaOMe, CH₂Cl₂/MeOH (71%); e. 7, TMSOTf, CH₂Cl₂ (92%); f. NaOMe (69%); g. 8, TBSOTf, CH₂Cl₂ (98%); h. NaOMe (83%); i 9, TMSOTF, CH₂Cl₂ (84%); j. (CH₂OH)₂, CSA, CH₃CN (81%); k. Cl₂P(O)OMe, Pyr. (88%); 1, TBAF, THF (61%); m, 11, tetrazole, CH₃CN; n. t-BuOOH, CH₃CN (84%, 2 steps); o. DBU, CH₂Cl₂; P. Na, NH₃ THF (75%, 2 steps). (AgOTf, silver trifluoromethanesulfonate; NIS, N-iodosuccinimide; CH₂Cl₂, dichloromethane, Et₂O, diethyl ether; NaOMe, sodium methoxide; MeOH, methanol; TMFOTf, trimethylsilyltrifluoromethane sulfonate; TBSOTf, tert-butyidimethylsilyl trifluoromethanesulfonate; CSA, camphorsulfonic acid; CH₃CN, acetonitrile; Cbz, carbobenzyloxy; Pyr, pyridine; TBAF, tetrabutylammonium fluoride; THF, tetrahydrofuran; DBU, 1,8-diazabicyclo[5,4,0]undec-7-ene; Obn, O-benzyl).

### DETAILED DESCRIPTION OF THE INVENTION

The present invention is predicated, in part, on the elucidation of the nature and mechanism of action of GPIs in the context of mediating cellular signalling. Specifically, it has been determined that GPI-related signalling events play a crucial role in integrin mediated cellular activity events. This determination now permits the rational design of therapeutic and/or prophylactic methods for treating conditions characterised by aberrant or unwanted integrin mediated cellular activity. Further, there is now facilitated the identification and/or design of agents which mimic or modulate the interaction of GPIs with integrins.

Accordingly, one aspect of the present invention is directed to a method for regulating integrin-mediated cellular activity, said method comprising modulating the functional interaction of a GPI with an integrin wherein inducing or otherwise agonising said interaction upregulates said cellular activity and inhibiting or otherwise antagonising said interaction downregulates said cellular activity.

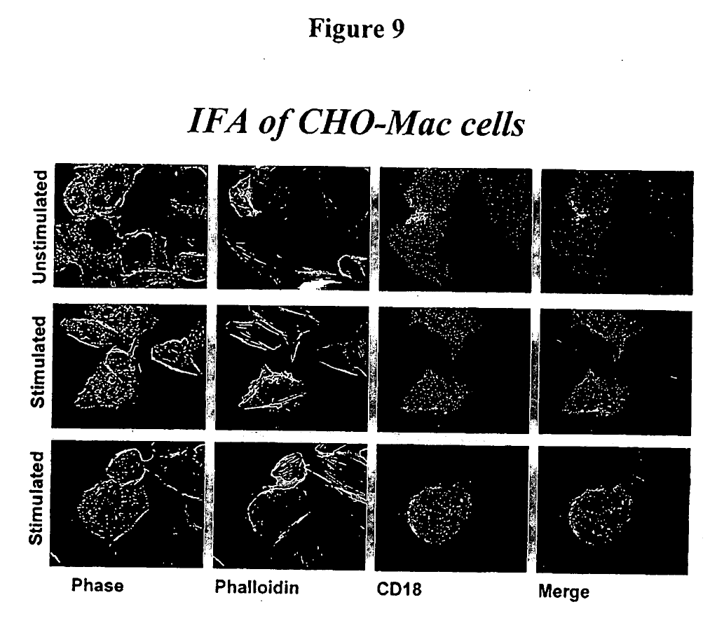

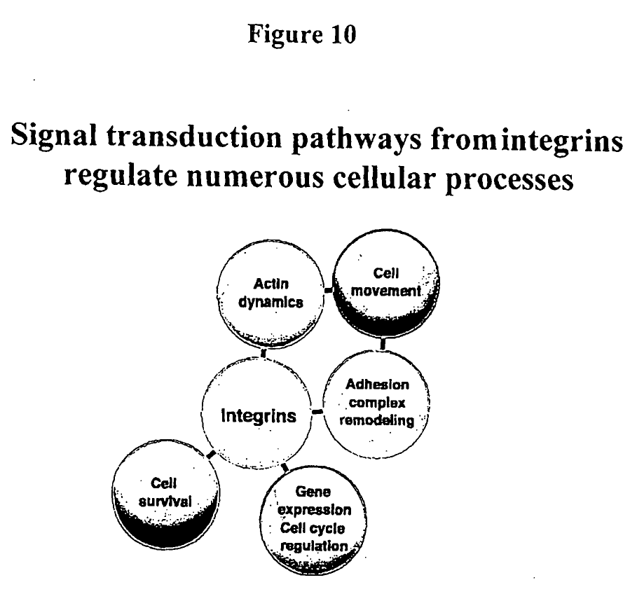

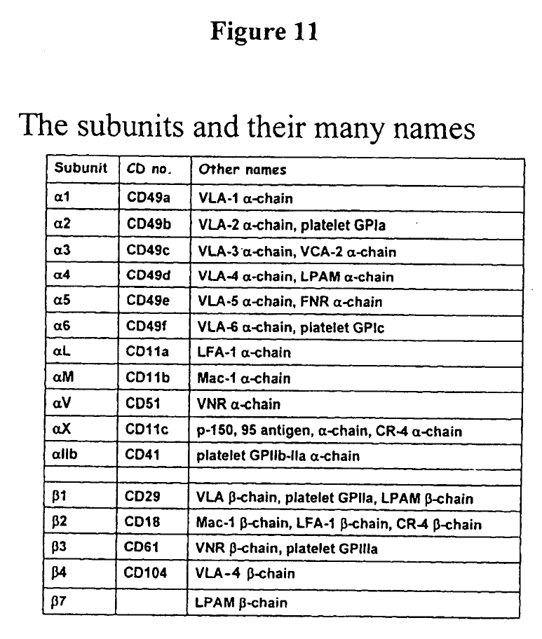

Without limiting the present invention to any one theory or mode of action integrins are a known family of transmembrane receptor proteins that function in a variety of cell-extracellular matrix and cell-cell interactions and are involved in cellular activities such as wound healing, cellular differentiation, extravasation and apoptosis (FIG. 9). Functional integrin is a heterodimer comprising non-covalently associated α and β transmembrane glycoprotein subunits (FIG. 10). 18 alpha and 8 beta subunits have been identified which combine to form some 24 complete integrins (FIG. 11). The structure between the α subunits is very similar. All contain 7 homologous repeats of 30-40 amino acids in their extracellular domain, spaced by stretches of 20-30 amino acids. The three or four repeats that are most extracellular contain sequences with cation-binding properties. The α chain is clearly involved in the ligand specificity because various β-1 α heterodimers have diverse ligand specificities. Some heterodimers have restricted pairing eg the β-4 with the α-6 subunit. Alternate splicing of some integrin messenger RNAs promotes further diversity. Integrin chains tend to have long extracellular domains which adhere to their ligands, and short cytoplasmic domains that link the receptors to the cytoskeleton of the cell. Integrins can bind an array of ligands. Common ligands are, for example, fibronectin and laminin, which are both part of the extracellular matrix. Both are recognized by multiple integrins. The integrins are grouped into families, for example, the VLA family, having the β₁ subunit; the LEUCAM family, which includes LFA-1 and Mac-1, having the β₂ subunit; and a group of other integrins having subunits β₃-β₈. The type of integrin expressed on the cell surface determines which molecules the cell will bind, and can be varied in different circumstances. For example, transforming growth factory-β increases expression of α₁β₁, α₂β₁, α₃β₁, and α₅β₁ integrins on fibroblasts and the αᵥβ₃ integrin on fibroblasts and osteosarcoma cells; interleukin-1β enhances β₁ expression on osteosarcoma cells; and in response to wounding, the keratinocyte, which normally expresses integrin α₆β₄, will express α₅β₁ (VLA-1, the fibronectin receptor) so that the keratinocyte will then migrate over fibronectin in the cell matrix, thus covering the wound.

Accordingly, reference to “integrin” should be understood as a reference to all forms of the members of the integrin family of molecules and to functional derivatives, homologues and mimetics thereof. Reference to “integrin” extends to both monomeric forms of the α and β subunits or homodimers or heterodimers of these subunits. Reference to “integrin” also extends to molecules comprising isoforms of the α and/or β subunits which arise from alternative splicing of the subject α and/or β subunit mRNA or functional mutants or polymorphic variants of these proteins. Preferably, said integrin is an αβ heterodimer (herein referred to as “αβ-integrin”).

There is therefore more particularly provided a method for regulating αβ-integrin-mediated cellular activity, said method comprising modulating the functional interaction of a GPI with said αβ-integrin wherein inducing or otherwise agonising said interaction upregulates said cellular activity and inhibiting or otherwise antagonising said interaction dowregulates said cellular activity.

Reference to “integrin-mediated cellular activity” should be understood as a reference to any one or more of the functional activities which a cell is capable of performing as a result of integrin-mediated stimulation. Still without limiting the present invention in any way, stimulation of integrins by any of the numerous types of GPIs results in the induction of a complex series of intracellular signalling events which ultimately lead to the induction of any one or more of a wide spectrum of cellular functional outcomes, the specificity of which outcome is largely dependent on the nature of both the GPI itself and the integrin to which it binds. In this regard, GPIs have been found to both initiate a unique functional outcome in their own right or to amplify or otherwise potentiate the signals generated by other unrelated molecules. For example, insulin signalling is a GPI-amplified signal. In this regard, many cells express a cell surface αβ-integrin which, upon being bound by its GPI ligand provides an ancillary signal concurrently with insulin binding to its receptor. Similarly, GPIs can be used to potentiate the actions of cytokines, hormones and growth factors. For example, the use of some cytokines at high concentrations, in order to achieve a requisite level of activity, can result in toxicity (nerve growth factor, for instance, is only useful in vivo when used in very high concentrations, which usually lead to serious side effects). However, the use of an appropriate GPI to provide an ancillary signal t the cell expressing the cytokine receptor in issue provides a means of potentiating the activity of the cytokine without the adverse toxic side effects which are consequent to achieving such increases in levels of activity with the use of high cytokine, hormone or growth factor concentrations. Although not intending to be limited to the exemplification provided herein, a chemically synthesised GPI based on the native structure of a neuronally derived GPI can provide a signal in neuronal tissue and potentiate the functional activity of NGF. Preferably, said integrin mediated cellular activity is potentiation of cytokine, hormone or growth factor signal transduction.

Reference to “cytokine” should be understood as a reference to any soluble protein hormone or hormone-like molecule. In this regard, reference to the classes of molecules which are sometimes alternatively referenced to as “hormones”, “growth factors”, “interleukins” or “colony stimulating factors”.

According to this preferred embodiment there is provided a method for potentiating cytokine signal transduction, said method comprising modulating the functional interaction of a GPI with an integrin, which integrin is expressed on the same cell as the receptor for said cytokine, wherein inducing or otherwise agonising said interaction potentiates said cytokine signal transduction.

Preferably, said cytokine is insulin.

Accordingly, there is provided a method for potentiating insulin signal transduction, said method comprising modulating the functional interaction of a GPI with an integrin, which integrin is expressed on the same cell as the receptor for said insulin, wherein inducing or otherwise agonising said interaction potentiates said insulin signal transduction.

Other examples of hormones, growth factors and cytokines that may be potentiated by GPI-integrin interactions are Insulin-Like Growth Factor-1, nerve growth factor, Epidermail Growth Factor, Brain-derived neurotrophic factor, neurotrophin-3, Thyroid Stimulating Hormone, Hepatic Growth Factor, Fibroblast Growth Factor, Transforming Growth Factor-β, Follicle Stimulating Hormone, Human Chorionic Gonadotrophin, Thyrotropin, Adrenocorticotropic Hormone (ACTH), Erythropoietin, Thrombopoietin, Interleukin-2 etc. Agonists of the GPI-integrin interaction may be used to potentiate the action of these molecules either in their natural state or supplied as pharmaceuticals. Conversely, antagonists my be used to modify or down-regulate the activity of these agents either in their natural state or supplied as pharmaceuticals. Modification includes a selective impact on one part or the whole of the downstream signalling process arising from interaction of said factor with its cognate receptor.

Again, without limiting the present invention to any one theory or mode of action, unlike many other cell-surface receptors, integrins generally bind ligands with a low affinity (10⁶-10⁹ liters/mole) and are usually present at 10-100 fold higher concentration on the cell surface. Integrins however can only bind their ligands when they exceed a certain minimal density at certain locations on the cell surface such as focal contacts and hemidesmosomes. When integrins are diffusely distributed over the cell surface, adhesion does not occur.

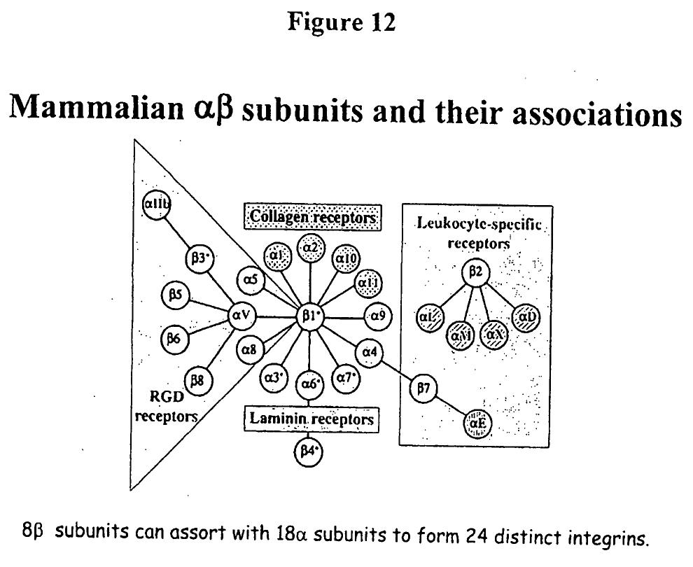

However, after appropriate stimuli integrins can cluster eg in focal contacts, their combined weak affinities giving rise to a spot on the cell surface with sufficient adhesive capacity for extracellular matrix binding. Integrin-ligand interactions are accompanied by clustering and activation of the integrins on the cell surface, which is also accompanied by the transduction of signals into intracellular signal transduction pathways that mediate a number of intracellular events. Signalling through integrins depends on the formation of focal adhesions, dynamic sites in which cytoskeletal and other proteins are concentrated which serve to compartmentalize many signalling pathways, where signalling cross-talk, regulation and integration can occur (FIG. 12).

GPIs are ubiquitous among eukaryotes, described from T. brucei, T. cruzi, Plasmodium, Leishmania, and Toxoplasma, as well as yeast, insect, fish and numerous mammalian sources (for recent reviews see McConville, M. J. and Ferguson, M. A., (1993) Biochem. J. 294:305 and Stevens, V. L. (1 995) Biochem. J. 310:361). GPIs consist of a conserved core glycan (Manα1-2Manα1-6Manα1-4GlcNH₂) linked to the 6-position of the myo-inositol ring of phosphatidylinositol (PI). GPIs are built up on the cytoplasmic face of the endoplasmic reticulum (ER) by the sequential addition of sugar residues to PI by the action of glycosyltransferases. The maturing GPI is then translocated across the membrane to the luminal side of the ER, whence it may be exported to the cell surface, free or in covalent association with proteins. The tetrasaccharide core glycan may be further substituted with sugars, phosphates and ethanolamine groups in a species and tissue-specific manner. GPI fatty acid moieties can be either diacylglycerols, alkylacylglycerols, monoalkylglycerols or ceramides, with additional palmitoylations or myristoylations to the inositol ring. The overall picture is of a closely related family of glycolipids sharing certain core features but with a high level of variation in fatty acid composition and side-chain modifications to the conserved core glycan.

Accordingly, reference to “GPI” should be read as including reference to all forms of GPI and derivatives, mutants, or equivalents thereof. An example of a GPI derivative is a GPI which lacks all or some of the lipidic domain. In accordance with the present invention, it has been determined that there exists specificity of signalling and pharmacological activity according to variation in structure in both the glycan and lipid domains of the GPI. For example, it has been shown that a chemically synthetic GPI based on the native structure of a neuronally-derived GPI can signal in neuronal tissue and potentiate the activity of NGF, but has little activity in macrophages, unlike GPIs with the simpler glycan (Ethanolamine-phosphate-5Manα1-2Manα1-6Manα1-GlcN1-6-inositol) which can activate macrophages. This indicates tissue specificity of action according to glycan composition. Similarly, GPIs with simple glycans but differing in fatty acid composition exhibit unique effects on target cells, establishing specificity of action according to lipid composition. Accordingly, tissue specificity of GPIs is provided by variation in glycan structure and diversity of signalling action according to lipid composition (lipid number, site of linkage to the inositol, chain length, degree of saturation, and type of linkage e.g. ether, ester or ceramide linkage). The specificity in action according to glycan compositions reflects the differential expression in distinct tissues of diverse integrin receptors.

Preferably, said GPI comprises both the glycan and lipidic domains (herein expressly referred to as an “intact GPI”). However, the present invention also extends to the use of GPI derivatives such as a GPI molecule which lacks the lipidic domain, since although lipid derived signals may be generated from lipidated GPIs following binding to integrins, GPI glycans alone binding to integrins are nevertheless able to generate some biologically important signals and cellular responses. In this regard, it should therefore be understood that reference to a GPI which lacks the lipid domain may be, herein, interchangeably referred to as a GPI derivative, as defined above, or a “GPI inositolglycan domain”. In this regard, reference to “GPI inositolglycan domain” or to “GPI derivative” (in the context of the non-lipidated GPI) should be read as including reference to all forms of GPI inositolglycan domains. The term “GPI inositolglycan” is used interchangeably with terms such as but not limited to “inositolglycan” (IG), “inositophosphoglycan” (IPG), “phosphoinositolglycan” (PIG), “phosphooligosaccharide” (POS) and the molecules described by these terms should be understood as “GPI inositolglycan” molecules. It should also be understood that reference to “GPI inositolglycan domain” includes reference to a GPI inositolglycan domain linked, bound or otherwise associated with non-inositolglycan molecules such as, but not limited to, the glycerol linker sequence which links the lipidic domain to the inositolglycan domain, a non-functional portion of the lipidic domain or an amino acid peptide. Similarly, a lipidated GPI may also be linked, bound or otherwise associated with non-GPI molecules, such as an amino acid sequence.

The present invention therefore more preferably provides a method for regulating αβ-integrin-mediated cellular activity, said method comprising modulating the functional interaction of an intact GPI with an integrin wherein inducing or otherwise agonising said interaction upregulates said cellular activity and inhibiting or otherwise antagonising said interaction downregulates said cellular activity.

In another preferred embodiment there is provided a method for regulating α⊕-integrin-mediated cellular activity, said method comprising modulating the functional interaction of a GPI inositolglycan domain with an integrin wherein inducing or otherwise agonising said interaction upregulates said cellular activity and inhibiting or otherwise antagonising said interaction downregulates said cellular activity.

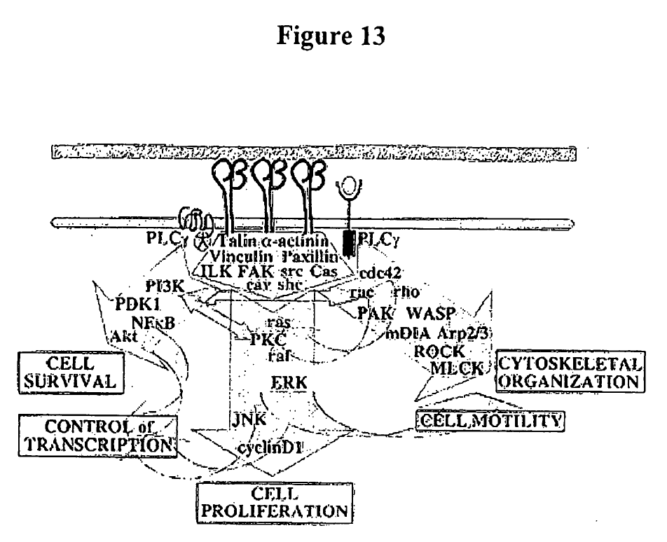

As detailed hereinbefore, there has been identified a two-signal mechanism induced by intact GPIs. In accordance with this “two-signal” mechanism (which does not preclude the occurrence of additional signals) the GPI inositolglycan domain binds to integrins which function as glycan-specific receptors (FIG. 13). These may either be originally located within “rafts” or may translocate to these structures after binding to GPI inositolglycan domains. There exists specificity in the glycan/integrin pair in that at physiologically and pharmacologically relevant concentrations not all GPI inositolglycan domains will bind to

all integrins. Modifications to GPI glycan structure causes greater or lower affinity binding to a range of integrins. Binding of the glycan initiates a signalling process involving src-kinases and members of the MAP kinase cascade. Following binding, the lipidated GPI will be hydrolysed by phospholipases leading to generation of lipidic second messengers which act both independently and in synergy with integrin-mediated signals to promote downstream metabolic and gene expression endpoints (FIG. 13).

The specificity of the interaction between a GPI inositolglycan domain and an integrin provides for an exquisitely precise mechanism for modulating the integrin-mediated cellular activity of a particular cell type. The combination of the 24 currently known integrins and the range of unique GPI structures provides for a large number of potential integrin/GPI glycan pairings, thereby providing for significant specificity of action by each of these unique integrin/GPI glycan combinations. Accordingly, in order to modulate a specific integrin-mediated cellular activity in accordance with the method of the present invention, it is necessary to know the structure of either the relevant GPI ligand of the integrin receptor of the cell in issue or the nature of the subject integrin molecule. To the extent that one is seeking to screen for an agonist, mimetic or antagonist of a specific GPI-integrin combination, it is preferable to know the structure of both the relevant integrin and its associated GPI. As detailed above, in light of the fact that the members of both the integrin and the GPI families have been widely identified and characterised, identifying functional GPI-integrin associations would be a matter of performing routine assays which screen for extent and affinity of binding between the glycan domain of a GPI and an integrin. In one example, this can be set up in a high throughput manner in order to rapidly identify these combinations. In this regard, the members of the integrin family have been well described in the literature. So too, the range of GPI inositolglycan domain structures are well known and include, for example, the following general structures:
(i) ethanolamine-phosphate-(Manα1,2)-Manα1,2Manα1,6Manα1,4GlcN-myo-inositol phosphoglycerol;
(ii) X₁-X₂-X₃-X₄-ethanolamine-phosphate-(Manα1,2)-Manα1,2Manα1,6Manα1,4GlcN-myo-inositol phosphoglycerol wherein X₁, X₂, X₃ and X₄ are any 4 amino acids;
(iii) EtN-P-[Mα2)Mα2 Mα6 Mα4Gα6Ino;
   EtN-P-[Mα2][G]Mα2 Mα6 Mα4Gα6Ino;
   EtN-P-[Mα2][X]Mα2 Mα6 Mα4Gα6Ino;
   EtN-P-[Mα2][EtN-P]Mα2 Mα6 Mα4Gα6Ino;
   EtN-P-Mα2 Mα6 Mα4G,
   Mα2 Mα6 Mα4G;
   EtN-P-Mα2 Mα6 M:
   EtN-P-[Mα2][G]Mα2 Mα6 Mα4G;
   EtN-P-[Mα2][X]Mα2 Mα6 Mα4G;
   EtN-P-[Mα2][EtN-P]Mα2 Mα6 Mα4G;
   Mα2 [Mα2][G]Mα2 Mα6 Mα4G;
   Mα2 [Mα2][X]Mα2 Mα6 Mα4G;
   Mα2 [Mα2][EtN-P]Mα6 Mα4G;
   Mα6 Mα4Gα6Ino;
   Mα2 Mα6 Mα4Gα6Ino;
   Mα2 [Mα2]Mα6 Mα4Gα6Ino;
   Mα2 [Mα2][G]Mα6 Mα4Gα6Ino;
   Mα2 [Mα2][X]Mα6 Mα4Gα6Ino;
   EtN-P-[Mα2][G]Mα2 Mα6 M;
   EtN-P-[Mα2][X]Mα2 Mα6 M;
   EtN-P-[Mα2][EtN-P][Mα2 Mα6 M;
   Mα2 [Mα2][G]Mα2 Mα6 M;
   Mα2 [Mα2][X]Mα2 Mα6 M;
   Mα2 [Mα2][EtN-P]Mα6 M;
   Mα2 Mα6 M;
   Mα6 Mα4G;
   EtN-P-[Mα2][G]Mα2 M;
   EtN-P-[Mα2][X]Mα2 M; or
   EtN-P-[Mα2][EtN-P]Mα2 M;
   wherein EtN is ethanolamine, P is phosphate, M is mannose, G is non-N-acetylated glucosamine, [G] is any non-N-acetylated hexosamine, Ino is inositol or inositol-phosphoglycerol, [X] is any other substituent, a represents α-linkages which may be substituted with β-linkages wherever required, and numeric values represent positional linkages which may be substituted with any other positional linkages as required.
   Preferably X is a sugar.

Examples of fully lipidated GPI compositions which may be used in the invention include but are not limited to:
i) Manα1-2Manα1-6Manα1-4GlcN1-6-inositol-phospho-diacyl-glycerol
ii) Manα1(Manα1-2)-2Manα1-6Manα1-4GlcN1-6-inositol-phospho-diacyl-glycerol
iii) Ethanolamine-phosphate-6Manα1-2Manα1-6Manα1-4GlcN1-6-inositol-phospho-diacyl-glycerol
iv) Ethanolamine-phosphate-6Manα1(Manα1-2)-2Manα1-6Manα1-4GlcN1-6-inositol-phospho-diacyl-glycerol
v) Ethanolamine-phosphate-6Manα1(Manα1-2)-2Manα1-6Manα1(EtN-phosphate)-4GlcN1-6-inositol-phospho-diacylglycerol
vi) Ethanolamine-phosphate-6Manα1(Manα1-2)-2Manα1(EtN-phosphate)-6Manα1(EtN-phosphate)-4GlcN1-6-inositol-phospho-diacylglycerol
vii) Ethanolamine-phosphate-6Manα1(Manα1-2)-2Manα1(EtN-phosphate)-6Manα1-4GlcN1-6-inositol-phospho-diacylglycerol
viii) Ethanolamine-phosphate-6Manα1(Manα1-2)-2Manα1(EtN-phosphate, GalNAcβ1-4)-6Manα1-4GlcN1-6-inositol-phospho-diacylglycerol
ix) Ethanolamine-phosphate-6Manα1(Manα1-2)-2Manα1(EtN-phosphate, Galβ-GalNAcβ1-4)-6Manα1-4GlcN1-6-inositol-phospho-diacylglycerol
x) Ethanolamine-phosphate-6Manα1(Manα1-2)-2Manα1(EtN-phosphate, Sialic acid-Galβ-GalNAcβ1-4)-6Manα1-4GlcN1-6-inositol-phospho-diacylglycerol
xi) Ethanolamine-phosphate-6Manα1-2Manα1(EtN-phosphate)-6Manα1-4GlcN1-6-inositol-phospho-diacylglycerol
xii) Ethanolamine-phosphate-6Manα1-2Manα1(EtN-phosphate, GalNAcβ1-4)-6Manα1-4GlcN1-6-inositol-phospho-diacylglycerol
xiii) Ethanolamine-phosphate-6Manα1-2Manα1(EtN-phosphate, Galβ-GalNAcβ1-4)-6Manα1-4GlcN1-6-inositol-phospho-diacylglycerol
xiv) Ethanolamine-phosphate-6Manα1-2Manα1(EtN-phosphate, Sialic acid-Galβ-GalNAcβ1-4)-6Manα1-4GlcN1-6-inositol-phospho-diacylglycerol
xv) Ethanolamine-phosphate-6Manα1(Manα1-2)-2Manα1(EtN-phosphate)-6Manα1 (EtN-phosphate)-4GlcN1-6-inositol-phospho-diacylglycerol
xvi) Number 1-15 above where the terminal ethanolamine phosphate is absent.
xvii) Numbers 1-16 above but also containing an acyl chain on the 2 position of inositol.
xviii) Numbers 1-17 above where the diacylglycerol contains fully saturated fatty acids.
xix) Numbers 1-18 above where the diacylglycerol contains unsaturated fatty acids in either or both the sn1 and sn2 positions.
xx) Numbers 1-19 above where instead of diacylglycerol is found any lipid or phospholipid including but not limited to alkylacylglycerol, monoalkylglycerol, ceramides etc.
xxi) Numbers 1-20 above where mannose residues are additionally substituted with any other hexoses, amino sugar, amino acids, phosphates, phosphonates, sulfates, sulfhydryls etc.
xxii) Numbers 1-21 above where the Man-3 residue i.e. the mannose residue furthest from the inositol in the conserved core glycan, is additionally linked to peptides of up to 6 amino acids long of any sequence.

α-linkages may be substituted with β-linkages wherever required (and vice versa), numeric values represent positional linkages which may be substituted with any other positional linkages as required. In all cases, lipids may be of any desired chain length and degree of saturation. Unsaturated bonds may be in any desired location on the lipid chain.

Any of these structures may be further modified by substituents of positive, negative or neutral charge such as phosphates, phosphoglycerol, hexosamines, amino acids, thiols etc in any position and with any type of linkage. These structures may be further modified by addition of any number of amino acids for the purpose of providing a linkage sequence to the lipidic domain.

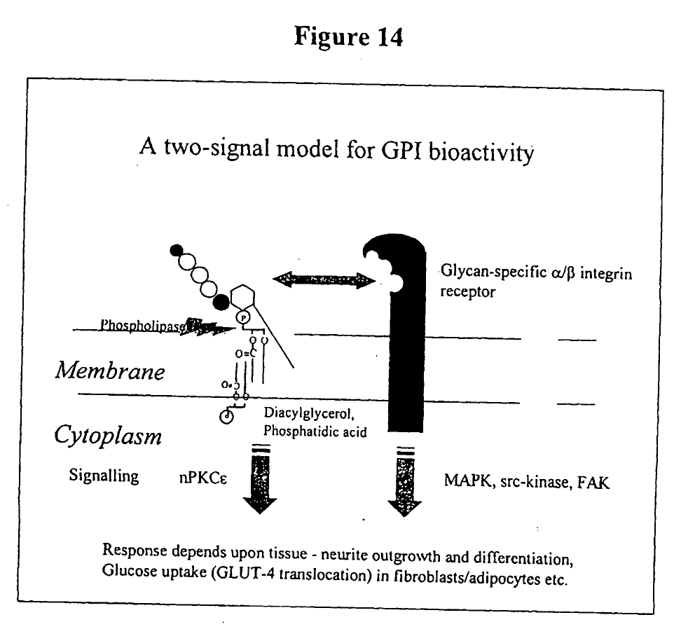

Reference to “derivative” herein should be understood to encompass, in one preferred embodiment, a GPI inositolglycan domain derivative wherein the terminal inositol-phosphoglycerol is substituted with inositol-1,2 cyclic-phosphate. Without limiting the present invention in any way, such a substitution is a characteristic outcome where certain forms of chemical synthesis are utilised, such as that exemplified in FIG. 14. For example, said GPI inositolglycan may exhibit the structure:

EtN-P-(Manα1,2)-6Mα1, 2Mα1, 6Manα1, 4GlcNH₂α1-myo-inositol-1,2 cyclic-phosphate wherein EtN is ethanolamine, P is phosphate and M is mannose.

NH₂—CH₂—CH₂—PO₄-(Manα1-2) 6Manα1-2 Manα1-6Manα1-4GlcNH₂-6myo-inositol-1,2 cyclic-phosphate

It should be understood that non-N-acetylated hexosamine includes glucosamine or any other nitrous-acid labile substituent. It should be further understood that any of these structures may be further modified by substituents including, but not limited to, of positive, negative or neutral charge such as phosphates, phosphoglycerol, hexosamines, amino acids or thiols in any position and with any type of linkage.

Without limiting the present invention in any way, focal adhesion kinase (FAK) is a tyrosine kinase which is associated with integrins and is commonly found in integrin-mediated focal adhesions. Upon activation and phosphorylation of FAK, this kinase may phosphorylate other signalling proteins in a signal transduction cascade. Paxillin, involved in cytoskeletal reorganization, is a target of FAK. One consequence of FAK activation is rapid cytoskeletal rearrangement. Activation of mitogen activated protein kinase (MAPK) occurs after integrin-ligand binding (RGD peptides, fibronectin, laminin), resulting in the translocation of MAPK from the cytoplasm to the nucleus. MAPK can also be activated by integrin linked kinase (ILK) in a FAK independent pathway. Induction of tyrosine phosphorylation of phospholipase C-gamma (PLC-gamma) and its recruitment to focal adhesions has been reported upon beta2-integrin activation. Activation of PLC-gamma, results in the hydrolysis of the phospholipid phosphatidylinositol diphosphate (PIP2) into diacyl glycerol (DAG) and inositol triphosphate (IP3). DAG is an activator protein kinase C (PKC), while IP3 mediates the release of calcium from mitochondrial calcium stores.

As detailed hereinbefore, intact GPIs have now been determined to provide a dual signal. Specifically, the GPI inositolglycan domain, in addition to providing cellular specificity by virtue of the unique glycan-integrin associations which have been determined to occur in the context of this invention, initiates a signalling process involving src kinases and members of the MAP kinase cascade. Following glycan binding, the lipidated GPI may be hydrolysed by phospholipases to generate lipidic second messengers which act both independently and in synergy with integrin-mediated signals to promote downstream metabolic and gene expression endpoints. Nevertheless, although intact GPIs are the preferred means of delivering a signal due to the dual signalling which is provided by the glycan and lipid domains of an intact GPI, it should be understood that even the GPI inositolglycan domain, alone, is able to bind to an integrin and, via a single step signal, deliver a biologically important signal which modulates an integrin mediated cellular function.

In this regard, reference to modulation of the “functional interaction” of a GPI with its integrin receptor should be understood as a reference to modulating the functional outcome of this interaction, that is, the induction of one or more signals. This will generally be achieved by modulating the physical interaction between the GPI and the integrin. However, it should also be understood to extend to modification of the functional outcome by other means. For example, signalling via the lipidic domain of an intact GPI is facilitated via its hydrolysis. Accordingly, modulation of this hydrolysis event provides an alternative means of modulating the functional outcome of GPI-integrin interaction. In a preferred embodiment, said functional interaction is a physical interaction.

Much of the bioactivity of intact GPIs result from the activation of lipid-dependent kinases by the GPI-derived lipids eg. Activation of PKC by the GPI-derived diacylglycerols, and activation of the sphingomyelinase pathway by GPI-derived ceramides. Accordingly, it is possible to use alternative pathways to the activation of the lipid-dependent pathway in conjunction with the inositolglycan, as a route to achieve desirable biochemical and pharmacological properties eg. fully lipidated GPI containing diacylglycerol is substantially more potent as an insulin-mimetic agent than IPG alone, as shown, and this activity at these concentrations of GPI can be blocked by PKC antagonists. However, the insulin-mimetic activity of the IPG can accordingly be boosted by the addition of phorbol esters which cause the activation of PKC by another route. Similarly, the activation of macrophages by GPI depends upon the presence of diacylglycerol, and IPG alone is relatively ineffectual. However, IPG with phorbol ester can activate macrophages, and indeed IPG synergizes with other agonists that act through PKC eg interferon-γ. Accordingly, the use of IPGs and GPIs together with known PKC- or sphingomyelinase-activating agents or other hormones, cytokines or growth factors that activate one or other of the various relevant sphingomyelinase or PKC isoforms falls within the scope of the present invention.

Elucidation of both the role of GPI and integrin in the context of integrin-mediated cellular activity and the nature of the interaction between these two molecules now provides a mechanism for regulating integrin-mediated cellular activity. By “regulated” is meant upregulated or dowuregulated. For example, antagonising the interaction of a GPI with an integrin provides a means of downregulating or abrogating the occurrence or degree of an integrin-mediated cellular activity, for example downregulating the catalysis of conversion of a native protein to an aberrant form, as is induced to occur by prions (these being GPI proteins). Conversely, to the extent that an integrin-mediated cellular activity is desirable, the method of the present invention now facilitates upregulation of such activity via agonism of a GPI/integrin interaction. For example, potentiation of cytokine signalling, such as insulin signal transduction.

Reference to “inducing or otherwise agonising” should be understood as a reference to:
(i) inducing the interaction of a GPI with an integrin in order to effect or potentiate integrin-mediated cellular activity; or
(ii) upregulating, enhancing or otherwise agonising a GPI/integrin interaction subsequently to its initial induction, for example agonising the hydrolysis step which occurs subsequently to binding of the lipid domain of a GPI or increasing the affinity of a GPI binding to its integrin receptor molecule.

Conversely, “inhibiting or otherwise antagonising” the interaction of a GPI with an integrin is a reference to:
(i) preventing the interaction of a GPI with an integrin; or
(ii) antagonising an existing interaction of a GPI with an integrin such that it is ineffective or less effective (for example, reducing the binding affinity of these two molecules).

It should be understood that modulation of the interaction between a GPI and an integrin (either in the sense of upregulation or downregulation) may be partial or complete. Partial modulation occurs where only some of the GPI/integrin interactions which would normally occur on a given cell are affected by the method of the present invention (for example, the method of the present invention is applied to a cell for only part of the time that the cell is undergoing integrin-mediated stimulation or the agent which is contacted with the subject cell is provided in a concentration insufficient to saturate the intracellular GPI/integrin interactions) while complete modulation occurs where all GPI/integrin interactions are modulated.

Modulation of the interaction between a GPI and an integrin may be achieved by any one of a number of techniques including, but not limited to:
(i) introducing into a subject a proteinaceous or non-proteinaceous molecule which either agonises or antagonises the interaction between a particular GPI and integrin (for example, introducing a soluble version of the relevant integrin molecule will competitively bind GPI, thereby making GPI unavailable for binding to the cell surface integrin molecules);
(ii) introducing into a subject the GPI of interest or derivative, mimetic or equivalent thereof;
(iii) inducing up or downregulation of the relevant integrin receptor molecule thereby effectively modulating the degree of signalling which is induced by a GPI molecule. Such regulation of integrin receptor expression can be achieved by:
   modulating the transcription and/or translation of the gene encoding said integrin
   introducing into the relevant cell population a nucleic acid molecule encoding the relevant integrin or a derivative, homologue or analogue thereof.

Reference to “agent” should be understood as a reference to any proteinaceous or non-proteinaceous molecule which modulates the interaction of a GPI with an integrin and includes, for example, the molecules detailed in points (i)-(iii), above. The subject agent may be linked, bound or otherwise associated with any other proteinaceous or non-proteinaceous molecule. For example, it may be associated with a molecule which permits its targeting to a localised region.

Said proteinaceous molecule may be derived from natural, recombinant or synthetic sources including fusion proteins or following, for example, natural product screening. Said non-proteinaceous molecule may be derived from natural sources, such as for example natural product screening or may be chemically synthesised. For example, the GPI inositolglycan domain may be synthesised in accordance with the methodology detailed in FIG. 14.

The present invention contemplates chemical analogues of said GPI or integrin capable of acting as agonists or antagonists of the GPI/integrin interaction. Chemical agonists may not necessarily be derived from said GPI or integrin but may share certain conformational similarities. Alternatively, chemical agonists may be specifically designed to mimic certain physiochemical properties of said GPI or integrin. Antagonists may be any compound capable of blocking, inhibiting or otherwise preventing said GPI and integrin from interacting. Antagonists include monoclonal antibodies specific for said GPI or integrin, or parts of said GPI or integrin, and antisense nucleic acids which prevent transcription or translation of integrin genes or mRNA in the subject cells. Modulation of expression may also be achieved utilising antigens, RNA, ribosomes, DNAzymes, RNA aptamers, antibodies or molecules suitable for use in co-suppression. Screening methods suitable for use in identifying such molecules are described in more detail hereinafter.

Said proteinaceous or non-proteinaceous molecule may act either directly or indirectly to modulate the interaction of a GPI with an integrin. Said molecule acts directly if it associates with the GPI or integrin molecules. Said molecule acts indirectly if it associates with a molecule other than the GPI or integrin, which other molecule either directly or indirectly modulates the interaction of the GPI with the integrin. Accordingly, the method of the present invention encompasses regulation of the GPI/integrin interaction via the induction of a cascade of regulatory steps. Preferably, said molecule acts directly.

Screening for the modulatory agents hereinbefore defined can be achieved by any one of several suitable methods including, but in no way limited to, contacting a cell comprising the integrin gene or functional equivalent or derivative thereof with an agent and screening for the modulation of integrin protein production or functional activity, modulation of the expression of a nucleic acid molecule encoding integrin or modulation of the activity or expression of an integrin-mediated functional outcome. Detecting such modulation can be achieved utilising techniques such as Western blotting, electrophoretic mobility shift assays and/or the readout of reporters of integrin activity such as luciferases, CAT and the like.

It should be understood that the integrin gene or functional equivalent or derivative thereof may be naturally occurring in the cell which is the subject of testing or it may have been transfected into a host cell for the purpose of testing. Further, the naturally occurring or transfected gene may be constitutively expressed—thereby providing a model useful for, inter alia, screening for agents which down regulate integrin activity, at either the nucleic acid or expression product levels, or the gene may require activation—thereby providing a model useful for, inter alia, screening for agents which up regulate integrin expression. Further, to the extent that an integrin nucleic acid molecule is transfected into a cell, that molecule may comprise the entire integrin gene or it may merely comprise a portion of the gene such as the portion which regulates expression of the integrin product. For example, the integrin promoter region may be transfected into the cell which is the subject of testing. In this regard, where only the promoter is utilised, detecting modulation of the activity of the promoter can be achieved, for example, by ligating the promoter to a reporter gene. For example, the promoter may be ligated to luciferase or a CAT reporter, the modulation of expression of which gene can be detected via modulation of fluorescence intensity or CAT reporter activity, respectively.

In another example, the subject of detection could be a downstream integrin regulatory target or functional outcome, rather than integrin itself. Yet another example includes integrin binding sites ligated to a minimal reporter. This is an example of an indirect system where modulation of integrin expression, per se, is not the subject of detection. Rather, modulation of the molecules or functional activities which integrin mediated signalling regulates are monitored.

These methods provide a mechanism for performing high throughput screening of putative modulatory agents such as the proteinaceous or non-proteinaceous agents comprising synthetic, combinatorial, chemical and natural libraries. These methods facilitate the detection of agents which modulate integrin expression or modulate the interaction of a GPI molecule with an integrin (this latter objective can be achieved, for example, by introducing GPI into the screening assays described above and detecting either agonism or antagonism of GPI-integrin binding or functional outcome). These assays can also be applied to screening populations of GPI molecules in order to identify the GPI ligand for a specific integrin molecule. As described hereafter, these assays provide the basis for high throughput methods of screening for agonists/antagonists of GPI/integrin binding and for identifying suitable GPIs or GPI mimetics for upregulation of integrin mediated cellular activity.

The term “expression” refers to the transcription and translation of a nucleic acid molecule. Reference to “expression product” is a reference to the product produced from the transcription and translation of a nucleic acid molecule.

In such situations, functional outputs may not be required to be assessed in the first instance and one can instead simply screen for the occurrence or modulation of the physical interactions between GPIs and integrins. This may be achieved, for example, by binding one of these molecules to a solid phase and thereafter screening populations of putative binding partners for their capacity to bind to the immobilised GPI or integrin. Again this provides a particularly useful means for identifying the GPI ligands for individual integrin molecules or for identifying lead compounds which can be thereafter analysed in the context of the functional impact of their interaction with GPI or integrin molecules.

“Derivatives” of the molecules herein described (for example GPI or integrin or other proteinaceous or non-proteinaceous agents) include fragments, parts, portions or variants from either natural or non-natural sources. Non-natural sources include, for example, recombinant or synthetic sources. By “recombinant sources” is meant that the cellular source from which the subject proteinaceous molecule is harvested has been genetically altered. This may occur, for example, in order to increase or otherwise enhance the rate and volume of production by that particular cellular source. Parts or fragments include, for example, active regions of the molecule. Derivatives of proteins may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterised by the removal of one or more amino acids from the sequence. Substitutional amino acid variants are those in which at least one residue in a sequence has been removed and a different residue inserted in its place. Additions to amino acid sequences include fusions with other peptides, polypeptides or proteins, as detailed above.

Derivatives also include fragments having particular epitopes or parts of the entire protein fused to peptides, polypeptides or other proteinaceous or non-proteinaceous molecules. For example, GPI or derivative thereof may be fused to a molecule to facilitate its targeting to a specific tissue. Analogues of the molecules contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Derivatives of nucleic acid sequences which may be utilised in accordance with the method of the present invention may similarly be derived from single or multiple nucleotide substitutions, deletions and/or additions including fusion with other nucleic acid molecules. The derivatives of the nucleic acid molecules utilised in the present invention include oligonucleotides, PCR primers, antisense molecules, molecules suitable for use in cosuppression and fusion of nucleic acid molecules. Derivatives of nucleic acid sequences also include degenerate variants.

A “variant” of GPI or integrin should be understood to mean molecules which exhibit at least some of the functional activity of the form of GPI or integrin of which it is a variant. A variation may take any form and may be naturally or non-naturally occurring. A mutant molecule is one which exhibits modified functional activity.

A “homologue” is meant that the molecule is derived from a species other than that which is being treated in accordance with the method of the present invention. This may occur, for example, where it is determined that a species other than that which is being treated produces a form of GPI which exhibits similar and suitable functional characteristics to that of the GPI which is naturally produced by the subject undergoing treatment.

Chemical and functional equivalents should be understood as molecules exhibiting any one or more of the functional activities of the subject molecule, which functional equivalents may be derived from any source such as being chemically synthesised or identified via screening processes such as natural product screening.

Chemical or functional equivalents or agonistic or antagonistic agents can be designed and/or identified utilising well known methods such as combinatorial chemistry or high throughput screening of recombinant libraries or following natural product screening.

For example, libraries containing small organic molecules may be screened, wherein organic molecules having a large number of specific parent group substitutions are used.

A general synthetic scheme may follow published methods (eg., Bunin B A, et al. (1994) Proc. Natl. Acad Sci. USA, 91:4708-4712; DeWitt S H, et al. (1993) Proc. Natl. Acad. Sci. USA, 90:6909-6913). Briefly, at each successive synthetic step, one of a plurality of different selected substituents is added to each of a selected subset of tubes in an array, with the selection of tube subsets being such as to generate all possible permutation of the different substituents employed in producing the library. One suitable permutation strategy is outlined in U.S. Pat. No. 5,763,263.

There is currently widespread interest in using combinational libraries of random organic molecules to search for biologically active compounds (see for example U.S. Pat. No. 5,763,263). Ligands discovered by screening libraries of this type may be useful in mimicking or blocking natural ligands or interfering with the naturally occurring ligands of a biological target. In the present context, for example, they may be used as a starting point for developing GPI analogues which exhibit properties such as more potent pharmacological effects. GPI or integrin or a functional part thereof may according to the present invention be used in combination libraries formed by various solid-phase or solution-phase synthetic methods (see for example U.S. Pat. No. 5,763,263 and references cited therein). By use of techniques, such as that disclosed in U.S. Pat. No. 5,753,187, millions of new chemical and/or biological compounds may be routinely screened in less than a few weeks. Of the large number of compounds identified, only those exhibiting appropriate biological activity are further analysed.

With respect to high throughput library screening methods, oligomeric or small-molecule library compounds capable of interacting specifically with a selected biological agent, such as a biomolecule, a macromolecule complex, or cell, are screened utilising a combinational library device which is easily chosen by the person of skill in the art from the range of well-known methods, such as those described above. In such a method, each member of the library is screened for its ability to interact specifically with the selected agent. In practising the method, a biological agent is drawn into compound-containing tubes and allowed to interact with the individual library compound in each tube. The interaction is designed to produce a detectable signal that can be used to monitor the presence of the desired interaction. Preferably, the biological agent is present in an aqueous solution and further conditions are adapted depending on the desired interaction. Detection may be performed for example by any well-known functional or non-functional based method for the detection of substances.

In addition to screening for molecules which mimic the activity of a specific GPI, it may also be desirable to identify and utilise molecules which function agonistically or antagonistically to the GPI-integrin binding in order to up or down-regulate the functional activity of integrin mediated cellular activity. The use of such molecules is described in more detail below. To the extent that the subject molecule is proteinaceous, it may be derived, for example, from natural or recombinant sources including fusion proteins or following, for example, the screening methods described above. The non-proteinaceous molecule may be, for example, a chemical or synthetic molecule which has also been identified or generated in accordance with the methodology identified above. Accordingly, the present invention contemplates the use of chemical analogues of GPI or integrin capable of acting as agonists or antagonists. Chemical agonists may not necessarily be derived from GPI or integrin but may share certain conformational similarities. Alternatively, chemical agonists may be specifically designed to mimic certain physiochemical properties of GPI or integrin. Antagonists may be any compound capable of blocking, inhibiting or otherwise preventing GPI or integrin from carrying out its normal biological functions. Antagonists include monoclonal antibodies specific for GPI or integrin or parts thereof.

In a most preferred embodiment, identification of integrins as GPI-receptors provides for the screening of combinatorial libraries and natural or synthetic products for receptor agonist activity where these activities reflect the biological properties of GPIs or IPGs eg. recombinant integrins either purified or expressed on the surface of cells may be used in assays involving multi-array screening methods for the measurement of binding of combinatorial libraries of carbohydrate or peptide composition or for the screening of a desired biological endpoint such as impact on cellular response. Such assays may also make use of plasmon resonance or similar methods for measuring the affinity for receptors of various candidates. Similarly, transfection of cells or animals with integrins and mutant versions allows the further identification of candidate variant IPG or GPI structures with specific properties of cell signalling and pharmacological usage.

Analogues of integrin or other proteinaceous modulatory agents contemplated herein include, but are not limited to, modifications to side chains, incorporating unnatural amino acids and/or derivatives during peptide, polypeptide or protein synthesis and the use of crosslinkers and other methods which impose conformational constraints on the analogues. The specific form which such modifications can take will depend on whether the subject molecule is proteinaceous or non-proteinaceous. The nature and/or suitability of a particular modification can be routinely determined by the person of skill in the art.

For example, examples of side chain modifications contemplated by the present invention include modifications of amino groups such as by reductive alkylation by reaction with an aldehyde followed by reduction with NaBH4; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 1.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl--aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2- | Nmbc | | |
| diphenyl-ethylamino)cyclopropane | | | |

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

The method of the present invention contemplates the modulation of cellular functioning both in vitro and in vivo. Although the preferred method is to treat an individual on vivo, it should nevertheless be understood that it may be desirable that the method of the invention be applied in an in vitro environment.

A further aspect of the present invention relates to the use of the invention in relation to the treatment and/or prophylaxis of disease conditions. Without limiting the present invention to any one theory or mode of action, the broad spectrum of integrin mediated cellular activities renders this molecule an integral functional component of every aspect of both healthy and disease state biological processes. Accordingly, the present invention provides a valuable tool for modulating aberrant or otherwise unwanted integrin mediated cellular activity.

Without limiting the present invention in any way, integrins are known to associate with a number of GPI-linked proteins on the surface of diverse leukocytes such as CD14, Fc gamma RIIIB and uPAR. These proteins are intimately involved in inflammatory responses. The association of these molecules with integrins is mediated specifically by binding of their associated GPI anchors with integrin lectin sites. This model is in contrast to the proposed models where the interaction results either from protein-integrin interactions or unique N-linked glycosylation on the protein, distinct to GPIs. Accordingly, inhibition of the interaction of integrins with GPIs by antagonists is useful in the treatment of clinical conditions associated with leukocyte receptor biology, in particular sepsis, arthritis and ischemia-reperfusion injury.

Thus the promotion or inhibition of GPI-integrin interactions is useful in (i) treatment of nerve, spinal cord or central nervous system damage secondary to trauma, autoimmune or metabolic change, including age-related memory loss and neurodegenerative disease, or post-ischaemic damage secondary to stroke or post-transplant complications; (ii) the treatment of hepatic damage caused by infection, alcohol/drug abuse, drug sensitivity, or autoimmunity; (iii) FGF and EGF-mimetic activities for the promotion of wound healing following surgery or trauma or tissue damage induced by ischaemia or autoimmunity; (iv) the treatment of a disease state involving adrenal atrophy eg tuberculosis; (v) for the promotion of haematopoiesis and the regulation of cell differentiation; (vi) for the treatment of cancers and neoplasias where GPIs with the appropriate lipid composition impart an appropriate apoptotic or cell death signal, or serve to downregulate cell growth.

Accordingly, another aspect of the present invention is directed to a method for the treatment and/or prophylaxis of a condition characterised by aberrant integrin-mediated cellular activity, said method comprising modulating the functional interaction of a GPI with an integrin wherein inducing or otherwise agonising said interaction upregulates said cellular activity and inhibiting or otherwise antagonising said interaction downregulates said cellular activity.

More particularly the present invention is directed to a method for the treatment and/or prophylaxis of a condition characterised by aberrant αβ-integrin mediated cellular activity, said method comprising modulating the functional interaction of a GPI with an integrin wherein inducing or otherwise agonising said interaction upregulates said cellular activity and inhibiting or otherwise antagonising said interaction downregulates said cellular activity.

In one preferred embodiment, said GPI is an intact GPI.

In another preferred embodiment, said GPI is a GPI inositolglycan domain.

Preferably, said modulation is effected via the administration of an agent as hereinbefore described.

Reference to “aberrant” cellular activity should be understood as a reference to overactive cellular activity, to biologically normal cellular activity which is inappropriate in that it is unwanted or to insufficient cellular activity. For example, neurodegenerative diseases which are characterised by prion infection are known to involve GPI-integrin mediated catalysis of the conversion of native protein to an aberrant form. Prions are all GPI-proteins. In such a situation, it is desirable to downregulate such activity. In yet another example, it is desirable to therapeutically potentiate insulin signal transduction in type II diabetes (or to prophylactically do so in patients predisposed to developing type II diabetes. In another example, it may be desirable to up- or downregulate the activity of a cytokine by modulating its potentiation via GPI signalling. This may be particularly useful in the context of inflammatory conditions.

Accordingly, in one preferred embodiment the present invention is directed to a method for the treatment and/or prophylaxis of type II diabetes, said method comprising modulating the functional interaction of a GPI with an αβ-integrin, which integrin is expressed on the same cells as the insulin receptor, wherein inducing or otherwise agonising said interaction potentiates insulin signal transduction.

In another preferred embodiment, the present invention is directed to a method for therapeutically and/or prophylactically treating a prion-related neurodegenerative condition, said method comprising modulating the functional interaction of said prion GPI with an αβ-integrin wherein antagonising said interaction downregulates prion related catalysis of the conversion of native proteins to an aberrant form.

In accordance with these preferred embodiments, said GPI is an intact GPI.

The term “mammal” as used herein includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Preferably, the mammal is human or a laboratory test animal. Even more preferably, the mammal is a human.

An “effective amount” means an amount necessary at least partly to attain the desired response, or to delay the onset or inhibit progression or halt altogether, the onset or progression of a particular condition being treated. The amount varies depending upon the health and physical condition of the individual to be treated, the taxonomic group of individual to be treated, the degree of protection desired, the formulation of the composition, the assessment of the medical situation, and other relevant factors. It is expected that the amount will fall in a relatively broad range that can be determined through routine trials.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

The present invention further contemplates a combination of therapies, such as the administration of the agent together with subjection of the mammal to other treatment regimes. For example, a patient suffering severe type II diabetes might be treated with a combination of the agent of the present invention and insulin.

Administration of the modulatory agent, in the form of a pharmaceutical composition, may be performed by any convenient means and will depend on the nature of the particular modulatory agent. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable.

Considering a patient, for example, from about 0.1 μg to about 1 μg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The modulatory agent may be administered in a convenient manner such as by the oral, intravenous (where water soluble), intraperitoneal, intramuscular, subcutaneous, intradermal or suppository routes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant.

In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. For example, the subject agent may be administered together with an agonistic agent in order to enhance its effects. By “sequential” administration is meant a time difference of from seconds, minutes, hours or days between the administration of the two types of molecules. These molecules may be administered in any order.

Another aspect of the present invention contemplates the use of an agent, as hereinbefore defined, in the manufacture of medicament for the treatment of a condition in a mammal, which condition is characterised by aberrant integrin-mediated cellular activity, wherein said agent modulates the interaction of a GPI with an integrin and wherein inducing or otherwise agonising said interaction up-regulates said cellular activity and inhibiting or otherwise antagonising said interaction down-regulates said cellular activity.

Preferably, said integrin is an αβ-integrin.

In one preferred embodiment, said GPI is an intact GPI.

In another preferred embodiment, said GPI is a GPI inositolglycan domain.

Even more preferably, said condition is type II diabetes and said modulation of integrin-mediated cellular activity is potentiation of insulin signal transduction or said condition is a prion induced neurodegenerative condition and said modulation of integrin-mediated cellular activity is down regulation of prion related catalysis of the conversion of native proteins to an aberrant form.

In yet another further aspect, the present invention contemplates a pharmaceutical composition comprising the modulatory agent as hereinbefore defined together with one or more pharmaceutically acceptable carriers and/or diluents. Said agents are referred to as the active ingredients.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding a modulatory agent. The vector may, for example, be a viral vector.

Yet another aspect of the present invention relates to the agent as hereinbefore defined, when used in the method of the present invention.

Still another aspect of the present invention provides a method for detecting an agent capable of modulating the interaction of a GPI with an integrin or its functional equivalent or derivative thereof said method comprising contacting a test system containing said GPI and/or integrin or its functional equivalent or derivative with a putative agent and screening for modulated functional interaction.

Reference to “GPI” and “integrin” should be understood as a reference to either the GPI or integrin expression product or to a portion or fragment of the GPI or integrin molecule, such as the GPI binding region of the integrin protein.

Reference to “test system” should be understood as a reference to any suitable in vitro or in vivo system which provides means for screening for agents which can modulate the interaction between a receptor and its ligand. Preferably, the system is an in vitro system which facilitates the high throughput screening of putative modulatory agents. In this regard, the test system may screen at either or both of the physical or functional levels. For example, it may screen only for modulation of the physical interaction of a GPI and an integrin or it may screen for modulation of the interaction based on a functional readout, such as modulation of the relevant integrin-mediated cellular activity. Such screening techniques have been hereinbefore described in detail. Nevertheless, it should also be understood that the “agent” which is the subject of detection by this method may be one which agonises or antagonises the interaction between a GPI and an integrin, by appropriately binding to one or both of these molecules, or it may be one which mimics or actually corresponds to the relevant GPI or integrin molecule. This latter aspect is particularly important in the context of screening panels of GPI and integrin molecules in order to precisely identify the GPIs which act as ligands for the various integrin receptors.

The present invention is further described by reference to the following non-limiting examples.

### EXAMPLES 1

There has been identified a two-signal mechanism provided by intact GPIs, (glycan plus fatty acids). In this minimal “two-signal” mechanism (which does not preclude additional signals) the GPI glycan binds to integrins which function as glycan-specific receptors (FIG. 13). These may either be originally located within “rafts” or translocate to these structures after binding to GPI glycans. There exists specificity in the glycan/integrin pair i.e. at physiologically and pharmacologically relevant concentrations, not all GPI glycans will bind to all integrins. Modifications to GPI glycan structure may cause greater or lower affinity binding to a range of integrins. Binding of the glycan initiates a signalling process involving src-kinases and members of the MAP kinase cascade. Following binding, a lipidated a GPI may also be hydrolysed by phospholipases to generate lipidic second messengers which act both independently and in synergy with integrin-mediated signals to promote downstream metabolic and gene expression endpoints (FIG. 13).

Although lipid derived signals may be generated from lapidated GPIs following binding to integrins, GPI glycans alone binding to integrins are able to generate at least some biologically important signals and cellular responses.

There have been established specificity of signalling and pharmacological activity according to variation in structure in both the glycan and lipid domains. For example, has been shown that a chemically synthetic GPI based on the native structure of a neuronally-derived GPI can signal in neuronal tissue and potentiate the activity of NGF, but has little activity in macrophages, unlike GPIs with the simpler glycan Ethanolamine-phosphate-6Manα1-2Manα1-6Manα1-4GlcN1-6-inositol can activate macrophages. This indicates tissue specificity of action according to glycan composition. Similarly, GPIs with simple glycans but differing in fatty acid composition have very different effects on target cells, establishing specificity of action according to lipid composition. The specificity in action according to glycan composition reflects the differential expression in distinct tissues of diverse integrin receptors.

The identification of integrins as GPI-receptors facilitates screening of combinatorial libraries and natural or synthetic products for receptor agonist activity where these activities reflect the biological properties of GPIs or IPGs eg. recombinant integrins either purified or expressed on the surface of cells are used in assays involving multi-array screening methods for the measurement of binding of combinatorial libraries of carbohydrate or peptide composition or for the screening of a desired biological endpoint such as impact on cellular response. Such assays make use of plasmon resonance or similar methods for measuring the affinity for receptors of various candidates. Similarly, transfection of cells or animals with integrins and mutant versions allows the further identification of candidate variant IPG or GPI structures with specific properties of cell signalling and pharmacological usage. Recombinant integrins containing the glycan-specific receptor domains are bound or fused to a reporter molecule capable of producing an identifiable signal, contacted with a chemical or biological sample putatively containing a ligand and screened for binding. In another example, the integrin or fragment or derivative containing the glycan binding site is immobilized and used for the affinity-purification of putative ligands. The binding of putative ligands to the receptor is also measured by plasmon resonance or similar methods.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Almeida, I. C. and Gazzinelli, R. T., (2001) J. Leukocyte Biol. 70, 467
Anderson R G, (1998) Annu. Rev. Biochem., 67, 199
Bohulslav J, et al, (1995) J. Exp. Med., 181, 1381
Bunin B A, et al. (1994) Proc. Natl. Acad. Sci. USA, 91.4708-4712.
Campos, M. A. et al., (2002) J. Immunol 167: 416
Chan B L, et al., (1989) Proc. Natl. Acad. Sci. USA, 86, 1756
Deeg M A, et al (I 993) Diabetes, 42, 1318
Devemy E, et al., (1994) Cell. Signal., 6, 523
DeWitt S H, et al. (1993) Proc. Natl. Acad Sci. USA, 90:6909-6913
Dorrestijn J, et al., (1998) Arch Physiol Biochem 106, 269
Eardley D D, et al., (1991) Science, 251, 78
Fanjul L F, et al., (1 993) J. Cell. Physiol., 155, 273
Ferguson M A J (1992) Biochem Soc Trans. 20, 243
Field M C, (1997) Glycobiology, 7, 161
Frick W, et al (1992) Biochemistry, 37, 13421
Hunter S J, et al (1998) Am J Med 105, 331
Kessler A et al (1998) Biochem J 330, 277
Lisanti M P, et al., (1995) Mol. Mem. Biol., 12, 121
Martiny L, et al., (1990) Cell. Signal, 2, 21
Mayor S, et al., (1994) Science, 264, 1948
Merida I, et al, (1990) Proc. Natl. Acad. Sci. USA, 87, 9421
McConville M J, et al., (1993) Biochem. J., 294, 305
Misek D E, et al (1992) J. Biol. Chem., 267, 16266
Misek D E, et al (1994) Endocrinology 135, 1869
Nystrom F H, et al., (1999) Cell Signal 11, 563
Pascare C. and Medzhitov R., (2003) Science 299, 1033
Repressa J, et al., (1991) Proc. Natl. Acad. Sci. USA, 88, 8016,
Saltiel A R, et al., (1983) Science 233, 967,
Saltiel A R, et al., (1987) Biochem. Biophys. Res. Commun., 149, 1084,
Saltiel A R, (1991) J. Bioenerg. Biomemb., 23, 29
Takeda K., et al (2002) Ann. Rev. Immunol. 21, 335
Thakkar J K, et al., (1990) J. Biol. Chem., 265, 5475
van der Kleij D et al., (2002) J Biol Chem 277, 48122
Vivien D, et al., (1993) J. Cell. Physiol., 155, 437

1. A method for regulating integrin-mediated cellular activity, said method comprising modulating the functional interaction of a GPI with an integrin wherein inducing or otherwise agonising said interaction upregulates said cellular activity and inhibiting or otherwise antagonising said interaction downregulates said cellular activity.

2. A method for the treatment and/or prophylaxis of a condition characterised by aberrant integrin-mediated cellular activity, said method comprising modulating the functional interaction of a GPI with an integrin wherein inducing or otherwise agonising said interaction upregulates said cellular activity and inhibiting or otherwise antagonising said interaction downregulates said cellular activity.

3. The method according to claim 1 or 2 wherein said integrin is αβ-integrin.

4. The method according to claim 3 wherein said αβ-integrin is a leukocyte-specific receptor.

5. The method according to claim 4 wherein said integrin receptor is one of:
(i) αLβ2;
(ii) αMβ2;
(iii) αxβ2;
(iv) αDβ2; or
(v) αEβ7.

6. The method according to claim 3 wherein said αβ-integrin receptor is a collagen receptor.

7. The method according to claim 6 wherein said integrin receptor is one of:
(ii) α1β1;
(ii) α2β1;
(iii) α10β1; or
(iv) α11β2.

8. The method according to claim 3 wherein said αβ-integrin receptor is a laminin receptor.

9. The method according to claim 8 wherein said laminin receptor is one of:
(i) α3β1;
(ii) α6β2;
(iii) α6β4; or
(iv) α7β2.

10. The method according to claim 3 wherein said αβ-integrin receptor is an RGD-receptor.

11. The method according to claim 10 wherein said RGD receptor is one of:
(i) α5β1;
αVβ1;
α8β1;
αVβ3;
αVβ5;
αVβ6;
αVβ8;
αIIbβ3;
αVβ3;
αVβ3;

12. The method according to claim 3 wherein said αβ-integrin receptor is one of:
(i) α4β2;
α4β7;
α9β7.

13. The method according to claim 3 wherein said GPI is an intact GPI.

14. The method according to claim 13 wherein said intact GPI is one of:
i) Manα1-2Manα1-6Manα1-4GlcN1-6-inositol-phospho-diacyl-glycerol
ii) Manα1(Manα1-2)-2Manα1-6Manα1-4GlcN1-6-inositol-phospho-diacyl-glycerol
iii) Ethanolamine-phosphate-6Manα1-2Manα1-6Manα1-4GlcN1-6-inositol-phospho-diacyl-glycerol
iv) Ethanolamine-phosphate-6Manα1(Manα1-2)-2Manα1-6Manα1-4GlcN1-6-inositol-phospho-diacyl-glycerol
v) Ethanolamine-phosphate-6Manα1(Manα1-2)-2Manα1-6Manα1(EtN-phosphate)-4GlcN1-6-inositol-phospho-diacylglycerol
vi) Ethanolamine-phosphate-6Manα1(Manα1-2)-2Manα1(EtN-phosphate)-6Manα1(EtN-phosphate)-4GlcN1-6-inositol-phospho-diacylglycerol
vii) Ethanolamine-phosphate-6Manα1(Manα1-2)-2Manα1(EtN-phosphate)-6Manα1-4GlcN1-6-inositol-phospho-diacylglycerol
viii) Ethanolamine-phosphate-6Manα1(Manα1-2)-2Manα1(EtN-phosphate, GalNAcβ1-4)-6Manα1-4GlcN1-6-inositol-phospho-diacylglycerol
ix) Ethanolamine-phosphate-6Manα1(Manα1-2)-2Manα1(EtN-phosphate, Galβ-GalNAcβ1-4)-6Manα1-4GlcN1-6-inositol-phospho-diacylglycerol
x) Ethanolamine-phosphate-6Manα1(Manα1-2)-2Manα1(EtN-phosphate, Sialic acid-Galβ-GalNAcβ1-4)-6Manα1-4GlcN1-6-inositol-phospho-diacylglycerol
xi) Ethanolamine-phosphate-6Manα1-2Manα1(EtN-phosphate)-6Manα1-4GlcN1-6-inositol-phospho-diacylglycerol
xii) Ethanolamine-phosphate-6Manα1-2Manα1(EtN-phosphate, GalNAcβ1-4)-6Manα1-4GlcN1-6-inositol-phospho-diacylglycerol
xiii) Ethanolamine-phosphate-6Manα1-2Manα1(EtN-phosphate, Galβ-GalNAcβ1-4)-6Manα1-4GlcN1-6-inositol-phospho-diacylglycerol
xiv) Ethanolamine-phosphate-6Manα1-2Manα1(EtN-phosphate, Sialic acid-Galβ-GalNAcβ1-4)-6Manα1-4GlcN1-6-inositol-phospho-diacylglycerol
xv) Ethanolamine-phosphate-6Manα1(Manα1-2)-2Manα1(EtN-phosphate)-6Manα1(EtN-phosphate)-4GlcN1-6-inositol-phospho-diacylglycerol
xvi) Number 1-15 above where the terminal ethanolamine phosphate is absent.
xvii) Numbers 1-16 above but also containing an acyl chain on the 2 position of inositol.
xviii) Numbers 1-17 above where the diacylglycerol contains fully saturated fatty acids.
xix) Numbers 1-18 above where the diacyl glycerol contains unsaturated fatty acids in either or both the sn1 and sn2 positions.
xx) Numbers 1-19 above where instead of diacylglycerol is found any lipid or phospholipid including but not limited to alkylacylglycerol, monoalkylglycerol, ceramides etc.
xxi) Numbers 1-20 above where mannose residues are additionally substituted with any other hexoses, amino sugar, amino acids, phosphates, phosphonates, sulfates, sulfhydryls etc.
xxii) Numbers 1-21 above where the Man-3 residue i.e. the mannose residue furthest from the inositol in the conserved core glycan, is additionally linked to peptides of up to 6 amino acids long of any sequence.
α-linkages may be substituted with β-linkages wherever required (and vice versa), numeric values represent positional linkages which may be substituted with any other positional linkages as required. In all cases, lipids may be of any desired chain length and degree of saturation. Unsaturated bonds may be in any desired location on the lipid chain.

15. The method according to claim wherein said GPI is the GPI inositolglycan domain.

16. The method according to claim 23 wherein said GPI inositolglycan domain is one of:
(i) ethanolamine-phosphate-(Manα1,2)-Manα1,2Manα1,6Manα1,4GlcN-myo-inositol phosphoglycerol;
(ii) X₁-X₂-X₃-X₄-ethanolamine-phosphate-(Manα1,2)-Manα1,2Manα1,6Manα1,4GlcN-myo-inositol phosphoglycerol wherein X₁, X₂, X₃ and X₄ are any 4 amino acids;
(iii) EtN-P-[Mα2]Mα2 Mα6 Mα4Gα6Ino;
EtN-P-[Mα2][G]Mα2 Mα6 Mα4Gα6Ino;
EtN-P-[Mα2][X]Mα2 Mα6 Mα4Gα6Ino;
EtN-P-[Mα2][EtN-P]Mα2 Mα6 Mα4Gα6Ino;
EtN-P-Mα2 Mα6 Mα4G;
Mα2 Mα6 Mα4G;
EtN-P-Mα2 Mα6 M;
EtN-P-[Mα2][G]Mα2 Mα6 Mα4G;
EtN-P-[Mα2][X]Mα2 Mα6 Mα4G;
EtN-P-[Mα2][EtN-P]Mα2 Mα6 Mα4G;
Mα2 [Mα2][G]Mα2 Mα6 Mα4G;
Mα2 [Mα2][X]Mα2 Mα6 Mα4G;
Mα2 [Mα2][EtN-P]Mα6 Mα4G;
Mα6 Mα4Gα6Ino;
Mα2 Mα6 Mα4Gα6Ino;
Mα2 [Mα2]Mα6 Mα4Gα6Ino;
Mα2 [Mα2][G]Mα6 Mα4Gα6Ino;
Mα2 [Mα2][X]Mα6 Mα4Gα6Ino;
EtN-P-[Mα2][G]Mα2 Mα6 M;
EtN-P-[Mα2][X]Mα2 Mα6 M;
EtN-P-[Mα2][EtN-P]Mα2 Mα6 M;
Mα2 [Mα2][G]Mα2 Mα6 M;
Mα2 [Mα2][X]Mα2 Mα6 M;
Mα2 [Mα2][EtN-P]Mα6 M,
Mα2 Mα6 M;
Mα6 Mα4G;
EtN-P-[Mα2l[G]Mα2 M;
EtN-P-[Mα2][X]Mα2 M; or
EtN-P-[Mα2][EtN-P]Mα2 M;
wherein EtN is ethanolamine, P is phosphate, M is mannose, G is non-N-acetylated glucosamine, [G] is any non-N-acetylated hexosamine, Ino is inositol or inositol-phosphoglycerol, [X] is any other substituent, α represents α-linkages which may be substituted with β-linkages wherever required, and numeric values represent positional linkages which may be substituted with any other positional linkages as required.

17. The method according to claim 16 wherein X is a sugar.

18. The method according to claim 3 wherein said GPI is a derivative exhibiting the structure:
EtN-P-(Manα1,2)-6Mα1, 2Mα1, 6Manα1, 4GlcNH₂α1-myo-inositol-1,2 cyclic-phosphate wherein EtN is ethanolamine, P is phosphate and M is mannose.

19. The method according to claim 3 wherein said GPI is a derivative exhibiting the structure:
NH₂—CH₂—CH₂—PO₄-(Manα1-2) 6Manα1-2 Manα1-6Manα1-4GlcNH₂-6myo-inositol-1,2 cyclic-phosphate.

20. The method according to claim 3 wherein said integrin-mediated cellular activity is cytokine, hormone or growth factor signal transduction.

21. The method according to claim 20 wherein said cytokine, hormone or grow%th factor is insulin.

22. The method according to claim 21 wherein said cytokine, hormone or growth factor is insulin-like growth factor-1.

23. The method according to claim 21 wherein said cytokine, hormone or growth factor is nerve growth factor.

24. The method according to claim 21 wherein said cytokine, hormone or growth factor is epidermal growth factor.

25. The method according to claim 21 wherein said cytokine, hormone or growth factor is brain-derived neurotrophic factor.

26. The method according to claim 21 wherein said cytokine, hormone or growth factor is neurotrophin-3.

27. The method according to claim 21 wherein said cytokine, hormone or growth factor is thyroid stimulating hormone.

28. The method according to claim 21 wherein said cytokine, hormone or growth factor is hepatic growth factor.

29. The method according to claim 21 wherein said cytokine, hormone or growth factor is fibroblast growth factor.

30. The method according to claim 21 wherein said cytokine, hormone or growth factor is transforming growth factor-β.

31. The method according to claim 21 wherein said cytokine, hormone or growth factor is follicle stimulating hormone.

32. The method according to claim 21 wherein said cytokine, hormone or growth factor is human chorionic gonadotrophin.

33. The method according to claim 21 wherein said cytokine, hormone or growth factor is thyrotropin.

34. The method according to claim 21 wherein said cytokine, hormone or growth factor is adrenocorticotropic hormone (ACTH).

35. The method according to claim 21 wherein said cytokine, hormone or growth factor is erythropoietin.

36. The method according to claim 21 wherein said cytokine, hormone or growth factor is thrombopoietin.

37. The method according to claim 21 wherein said cytokine, hormone or growth factor is interleukin-2.

38. The method according to any one of claims 4-37, wherein said regulation of integrin-mediated cellular activity is potentiation.

39. The method according to claim 38 wherein said potentiation is achieved by inducing the interaction of a GPI molecule with said integrin.

40. The method according to claim 38 wherein potentiation is achieved by inducing the interaction of a fully lipidated GPI containing diacylglycerol with said integrin.

41. The method according to any one of claims 4-37 wherein said modulation is upregulation of the interaction of a GPI with an integrin.

42. The method according to claim 41 wherein said upregulation is achieved by contacting said cell with a proteinaceous or non-proteinaceous molecule which agonises said interaction.

43. The method according to claim 41 wherein said upregulation is achieved by contacting said cell with GPI or derivative thereof.

44. The method according to claim 43 wherein said GPI or derivative thereof is the GPI molecule of any one of claims 13-19.

45. The method according to claim 41 wherein said upregulation is achieved by upregulating integrin cell surface expression.

46. The method according to claim 45 wherein said upregulation of integrin cell surface expression is achieved by upregulating transcription and/or translation of the gene encoding said integrin.

47. The method according to claim 45 wherein said upregulation of integrin cell surface expression is achieved by introducing into said cell a nucleic acid molecule encoding said integrin.

48. The method according to any one of claims 4-37 wherein said modulation is downregulation of the interaction of a GPI with an integrin.

49. The method according to claim 40 wherein said downregulation is achieved by contacting said cell with a proteinaceous or non-proteinaceous molecule which antagonises said interaction.

50. The method according to claim 49 wherein said antagonist is an antibody directed to GPI and/or integrin.

51. The method according to claim 49 wherein said antagonist is a soluble integrin molecule.

52. The method according to claim 48 wherein said downregulation is achieved by introducing into said cell a nucleic acid molecule which downregulates the transcription and/or translation of said integrin DNA.

53. The method according to claim 52 wherein said molecule is RNAi or antisense DNA.

54. The method according to claim 23 wherein said NGF activity is potentiated, said integrin is a neuronal integrin is:

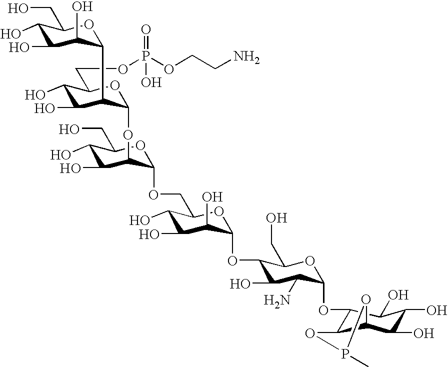

55. The method according to claim 3 wherein said cellular activity is macrophage activation, said activation is upregulated and said GPI is Ethanolamine-phosphate-5Manα1-2Manα1-6Manα1-GlcN1-6-inositol.

56. The method according to any one of claims 1-55 wherein said method is performed in vitro.

57. The method according to any one of claims 1-55 wherein said method is performed in vivo.

58. The method according to claim 2 wherein said condition is type II diabetes, said integrin molecule is expressed on the same cells as the insulin receptor and said interaction of GPI with said integrin is potentiated.

59. The method according to claim 58 wherein said GPI is intact.

60. The method according to claim 2 wherein said condition is a prion-related neurodegenerative condition, said GPI is prion GPI and said interaction of GPI with integrin is antagonised.

61. A pharmaceutical composition comprising the modulatory agent as hereinbefore defined together with one or more pharmaceutically acceptable carriers and/or diluents.

62. A method for detecting an agent capable of modulating the interaction of a GPI with an integrin or its functional equivalent or derivative thereof said method comprising contacting a test system containing said GPI and/or integrin or its functional equivalent or derivative with a putative agent and screening for modulated functional interaction.
